(19) United States
(12) Patent Application Publication
(10) Pub. No.: US 2026/0139283 A1
(43) Pub. Date: May 21, 2026

(54) PROCESS FOR THE REDUCTION OF AROMATIC IMINES WITH NOVEL IMINE REDUCTASES (IREDS)

(71) Applicant: Hoffmann-La Roche Inc., Little Falls, NJ (US)

(72) Inventors: Sarah Berger, Graz (AT); Serena Bisagni, Basel-City (CH); Francis Gosselin, San Mateo, CA (US); Christopher Grimm, Graz (AT); Hans Iding, Magden (CH); Irene Marzuoli, Basel (CH); Kurt Puentener, Kaiseraugst (CH); Joerg Schrittwieser, Graz (AT); Stephan Vrabl, Graz (AT)

(21) Appl. No.: 19/452,014

(22) Filed: Jan. 16, 2026

Related U.S. Application Data
(63) Continuation of application No. PCT/EP2024/070314, filed on Jul. 17, 2024.

(30) Foreign Application Priority Data
Jul. 18, 2023 (EP) 23185969.5

(51) Int. Cl. C12P 13/00; C12N 9/06; C12P 17/12

(52) U.S. Cl. CPC C12P 13/001; C12N 9/0028; C12P 17/12

(57) ABSTRACT

The present invention relates to a process for producing a chiral aromatic amine comprising the steps of
a) providing an aromatic imine, and
b) contacting the aromatic imine from step a) with an imine reductase,
   thereby stereoselectively reducing the aromatic imine with the imine reductase to a chiral aromatic amine and to novel imine reductases which are capable to catalyse the reaction.

### CROSS-REFERENCE TO RELATED APPLICATIONS

This application is a continuation of International PCT Application No. PCT/EP2024/070314 filed on Jul. 17, 2024, which claims priority to European Patent Application No. EP23185969.5 filed on Jul. 18, 2023, the disclosures of which are hereby incorporated by reference in their entirety.

### SEQUENCE LISTING

This application incorporates by reference in its entirety the material in the ST.26 XML file titled ROCHE-264 Sequence Listing.xml, which was created on Jan. 16, 2026 and is 39,402 bytes.

The present invention relates to a novel process for the preparation of chiral aromatic amines, comprising a) providing an aromatic imine and b) contacting the aromatic imine from step a) with an imine reductase, thereby stereoselectively reducing the aromatic imine with the imine reductase to a chiral aromatic amine.

The invention also relates to novel imine reductases which are capable of catalyzing the stereoselective reduction of an aromatic imine into a chiral aromatic amine.

Chiral aromatic amines are building blocks for the development of new therapeutic drug candidates. Indeed, stereoselective synthesis of chiral aromatic amines is a field that has been growing during the last decades because of the recurrence of these compounds in a wide variety of active pharmaceutical intermediates with different therapeutic targets.

In the art, different ways of chemical synthesis of chiral aromatic amines are known, such as the stereoselective reductive amination of a ketone and an amine, where an imine is transiently formed as an intermediate which is then reduced to the amine (Afanasyev et al. 2019).

Also known are reductive amination reactions from a ketone precursor and an aromatic amine with reductive aminases (Aleku et al. 2017, France et al. 2018, Roiban et al. 2017, Scheller et al. 2015).

However, these enzymes often fail to perform the reductive amination reaction, particularly when aromatic ketones and/or aromatic amines are involved.

The object of the invention therefore was to find a process which allows the preparation of chiral aromatic amines with high selectivity from a suitable precursor compound on a preparative scale.

It was found that the object of the invention could be achieved with the process as outlined below, which comprises:
a) providing an aromatic imine and
b) contacting the aromatic imine from step a) with an imine reductase, thereby stereoselectively reducing the aromatic imine with the imine reductase to a chiral aromatic amine.

The following definitions are set forth to illustrate and define the meaning and scope of the various terms used to describe the invention herein.

The term “polypeptide” or “enzyme” as used herein are macromolecular biological catalysts, usually proteins, and accelerate chemical reactions. The molecules upon which enzymes may act are substrates and the enzyme converts the substrates into products. Like all catalysts, enzymes increase the reaction rate by lowering its activation energy.

An “imine reductase/reductive aminase” is an enzyme capable of catalyzing the reduction of an imine into an amine group, most often into a secondary amine. The term imine reductases includes all enzymes capable of imine reduction, instead reductive aminases is a term that encompasses only the enzymes that are especially proficient at preforming direct reductive amination of the amine and the ketone, as it is believed that the imine is formed transiently in the active site of the enzyme, albeit also imine reductases are capable of reductive amination. Imine reductases and reductive aminases catalyze the reduction of the imine to the amine using the reducing equivalents provided by NADH or NADPH.

In accordance with the present invention, the proteins as defined herein in accordance with the present invention are used as an imine reductase. This means that the protein is contacted with a substrate containing an imine to be reduced under conditions allowing reduction and reduction takes place.

The term “unsubstituted alkyl” includes straight chain or branched chain alkyl groups of 1 to 6 C-atoms, such as methyl, ethyl, n-propyl, i-propyl, n-butyl, i-butyl, sec-butyl, t-butyl, pentyl and hexyl with its isomers. Preferred alkyl groups are those with 1 to 4 carbon atoms.

The term “substituted alkyl” includes the previously described alkyl chains, which may carry one or more substitutions selected from halogen, halogen alkyl, alkyl, hydroxy, aryl, alkyloxy, aryloxy or alkoxy carbonyl or aryloxy carbonyl, cyano, nitro.

The term “alkenyl”, includes straight chain or branched chain alkenyl groups of 2 to 6 C-atoms, which carry one or more double bonds in the chain. Typical alkenyl representatives are vinyl, or allyl.

The term “substituted alkenyl” includes the previously described alkyl or alkenyl chains, which may carry one or more substitutions selected from halogen, halogen alkyl, alkyl, hydroxy, aryl, alkyloxy, aryloxy or alkoxy carbonyl or aryloxy carbonyl, cyano, nitro.

The term “alkoxy” stands for an alkyl group as defined before which is attached to an oxygen atom. Typical example is methoxy.

The term “alkoxy carbonyl” stands for an alkyl group as defined before which is attached to a carbonyl group. Typical example is acetyl.

The term “aryl” includes phenyl, naphthyl, anthryl or phenanthryl, but preferably is phenyl.

The term “substituted aryl” includes the previously listed aryl groups, which may carry one or more substitutions selected from halogen, halogen alkyl, alkyl, hydroxy, aryl, alkyloxy, aryloxy or alkoxy carbonyl or aryloxy carbonyl, cyano, nitro.

The term “aryloxy” stands for an aryl group as defined before which is attached to an oxygen atom. Typical example is phenyl oxy.

The term “aryloxy carbonyl” stands for an aryl group as defined before which is attached to a carbonyl group. Typical example is benzoyl.

The term “heteroaryl” refers to an aromatic 5 to 6 membered monocyclic ring or 9 to 10 membered bicyclic ring which can comprise 1, 2 or 3 heteroatoms selected from nitrogen, oxygen and/or sulphur, such as pyridyl, pyridinyl, pyrazolyl, pyrimidinyl, benzoimidazolyl, quinolinyl and isoquinolinyl, thienyl, furyl, pyrrolyl, pyrazolyl, isoxazolyl, oxazolyl, thiazolyl or imidazolyl.

The term “halogen” includes F, Cl, Br, and I.

The term “cyano” signifies a CN substituent.

The term “nitro” stands for an NO₂ substituent.

The term “carbocycle” refers to a C₃₋₁₀-cycloalkane moiety, e.g. cyclohexane, which may be fused to aromatic ring systems. A typical example is tetrahydronaphthalene, where cyclohexane is fused with a benzene ring.

The term “heterocycle” refers to a C₃₋₁₀-cycloalkane moiety of which at least one carbon is replaced by a heteroatom selected from nitrogen, oxygen or sulfur. The heterocycle may be fused to aromatic ring systems.

The term “chiral” is used to describe a molecule which is not superimposable on its mirror image. Chiral molecules have a non-superimposable mirror image and exist in two distinct forms known as enantiomers.

The term “E or Z” configuration describes the relative orientation of two substituents on a double bond. An E configuration indicates that the two substituents are on opposite sides of the double bond, while a Z configuration indicates that the two substituents are on the same side of the double bond.

As outlined before the process comprises
a) providing an aromatic imine and
b) contacting the aromatic imine from step a) with an imine reductase, thereby stereoselectively reducing the aromatic imine with the imine reductase to a chiral aromatic amine.

In a preferred embodiment the process comprises stereoselectively reducing the aromatic imine with the imine reductase to a chiral primary or secondary aromatic amine.

“Providing an aromatic imine” in the context of the present invention preferably means that the aromatic imines are pre-formed, i.e. have been synthesized in advance and are provided as substrate for the reduction. Accordingly they do not occur as intermediates.

The preparation of the aromatic imines can be accomplished according to methods known in the art (Carlson et al, 1992, Yasukawa et al. 2022).

Suitable aromatic imines have the formula I

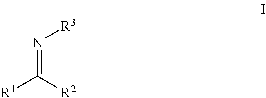

wherein
R¹ is substituted or unsubstituted aryl or heteroaryl,
R² is hydrogen, substituted or unsubstituted C₁₋₆-alkyl or C₂₋₆-alkenyl, substituted or unsubstituted C₁₋₆-alkoxy carbonyl or R¹ and R² together form a carbocycle and
R³ is hydrogen or substituted or unsubstituted aryl or heteroaryl, substituted or unsubstituted C₁₋₆-alkyl or substituted or unsubstituted C₁₋₆-alkoxy carbonyl
   and wherein
the substituents are selected from halogen, halogen C₁₋₆-alkyl, C₁₋₆-alkyl, hydroxy, aryl, C₁₋₆-alkyloxy, aryloxy or C₁₋₆-alkoxy carbonyl or aryloxy carbonyl, cyano or nitro.
   In a preferred embodiment
R¹ is substituted or unsubstituted aryl or heteroaryl,
R² is hydrogen, C₁₋₆-alkyl or C₁₋₆-alkoxy carbonyl or
R¹ and R² together form a carbocycle or heterocycle and
R³ is hydrogen or substituted or unsubstituted aryl or heteroaryl, substituted or unsubstituted C₁₋₆-alkyl or C₂₋₆-alkenyl,
   and wherein
the substituents are selected from C₁₋₆-alkyl, C₁₋₆-alkoxy or aryl.
   In a still further embodiment
R¹ is substituted or unsubstituted phenyl, naphthyl or an aromatic 5 to 6 membered monocyclic ring or 9 to 10 membered bicyclic ring which can comprise 1, 2 or 3 heteroatoms selected from nitrogen, oxygen and/or sulphur,
R² is hydrogen, C₁₋₆-alkyl or C₁₋₆-alkoxy carbonyl or
R¹ and R² together form a C₃₋₁₀-carbocycle or heterocycle, which can be fused to an aromatic ring and
R³ is hydrogen or substituted or unsubstituted phenyl, naphthyl or an aromatic 5 to 6 membered monocyclic ring or 9 to 10 membered bicyclic ring which can comprise 1, 2 or 3 heteroatoms selected from nitrogen, oxygen and/or sulphur, substituted or unsubstituted C₁₋₆-alkyl or C₂₋₆-alkenyl,
   and wherein
the substituents are selected from C₁₋₆-alkyl, C₁₋₆-alkoxy or aryl.

The aromatic imines of formula I may occur in the E or Z configuration.

Preferably the concentration of the aromatic imine is from 2 mM to 1000 mM, more preferably from 20 mM to 500 mM, and most preferably from 50 mM to 300 mM.

The stereoselective reduction in step b) as a rule takes place in the presence of a cofactor regeneration system comprising a cofactor and an enzyme capable of reducing such cofactor.

In a preferred embodiment the cofactor is NADP⁺ or NAD⁺ and the enzyme capable of reducing the cofactor is phosphite dehydrogenase or glucose dehydrogenase.

Preferably the cofactor is NADP⁺ and the enzyme capable of reducing the cofactor is phosphite dehydrogenase or glucose dehydrogenase.

The imine reductase can be applied as cell free extract either as a solution or as lyophilisate.

In a preferred embodiment, the reduction takes place in an aqueous environment having a pH of 5.0 to 10.0, preferably 6.5 to 8.5 at a temperature of 5° C. to 50° C., preferably in the range of 20° C. to 40° C., at a sodium phosphite or glucose concentration of from 100 to 1 M, preferably from 100 mM to 500 mM and at a phosphite dehydrogenase or glucose dehydrogenase concentration of from 0.1 g/L to 10 g/L, preferably from 0.1 g/L to 8 g/L with the aromatic imine, concentration of from 2 mM to 1000 mM, preferably from 20 mM to 500 mM.

As a rule a cosolvent selected either from polar protic solvents such as lower aliphatic alcohols, like methanol, dipolar aprotic solvents, like dimethylsulfoxide, Cyrene™ or non-polar solvents, like cyclohexane is applied.

Contacting/incubation time can vary from about 5 minutes to many hours, preferably from about 1 h to 100 h, such as 4 h to 50 h.

However, the incubation time can depend upon the assay format, volume of solution, concentrations of substrates and catalysts, the amount of aromatic imine to be reduced and the like. Those skilled in the art are familiar on how to apply the optimal incubation time.

Likewise for the addition of the aromatic imine, which can happen at the beginning of the reaction or in particular doses at different time points during the incubation period.

At the end of the reaction, the chiral aromatic amine is as a rule isolated by increasing the pH of the reaction e.g. by addition of an alkali carbonate base, followed by liquid-liquid extraction with a suitable organic solvent such as ethyl acetate. Subsequently, the solvent can be evaporated under reduced pressure and the chiral aromatic amine can be is isolated.

The resulting chiral aromatic amine has the formula II

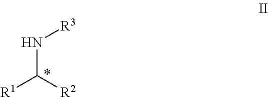

wherein R¹, R² and R³ are as defined before and wherein the same preferences apply.

The enzymes capable of catalyzing the stereoselective reduction of an aromatic imine into a chiral aromatic amine can be selected from imine reductases, which have a sequence having at least 80%, preferably at least 90%, more preferably at least 95%, even more preferably at least 98% sequence identity to any one of SEQ ID NOs. 1 to 33.

(IR00005)
SEQ ID 1
MHHHHHHKTVAVIGLGQMGTTLARLFIEAGMQVRVWNRTRSKAEPLASRGAIVA
ATAAAAMADAEAVVICVHDYRATHDILSDVAVKSALKGKLLLQLTTGSPQDARDM
AELAARIGAGYLDGALQVAPEQMGQPDTTVLVSGSGEDHALARELLAVLGGNVV
YLGEDVAAAATMDLATLSYVYGASMGFFQGAALAQAEGLDVGVYGGIVEAMSP
SFGAFLRHEGNVIDNGDYAVSQSPLSISIDATGRIEQAMRQKGLRSELPSLIARLL
RDAEEAGYGNEEFAAVAKILRGAAEPAPVR
(IR00010)
SEQ ID 2
MHHHHHHTTTPTVTVLGLGPMGQALSRALLDAGHTVTVWNRTESKAQALRDRG
ALSAPTPAAAIAASDLALVNVVDHDAVDAILTAAGDAPAGRTVIGLSSDTPDRARR
TAKLVGNVGGRYLDGAIMTPIDTIGTRGASILFAGPQALFDEHRGVLDTLGQLTW
VGEDHGRAAAFDMALLDLFWTSVGGFGHALMVARANGIEPSELMPHAHGIVGIL
SPIFTEVAQRVEDDRHSDASASVSSVASSVRHLIAASREAGVDAGLLEAFRGYVD
ATVAAGHGDDEISRIASEMTTLTRG
(IR00014)
SEQ ID 3
MHHHHHHTNNATPVSILGLGLMGQALARAFLKAGHPTTVWNRTPGKADQLMAE
GAQVAPTAAEAIDASSLTVICVSDYPAMYELLDASDLAGTTLLNLTSGDSAQARQ
AARWAEQRGAHYLDGAIMAIPQAIGTDDAVILISGAQADADAHRPTLEALGTLTYL
GADHGLASLYDVAGLAMMWSVLNAWLQGTALLRTAGVDAATFAPFAQQMAAG
VAGWLPGHAQEIDAGSFATEVASLDTHVRTMDHLIEECEAAGINAELPRLIKSMA
DRSLAAGHGAASYSVLIEEFAKPA
(IR00015)
SEQ ID 4
MHHHHHHMKAPVTVVGLGPMGKAMAETFLKNGHPTTVWNRTASKAAPLVEQG
ATLAATPDDALAASGLVVISQTDYKAMYDSLDGAEMKGRVLVNLSSGSPDELRR
AAEWAAGKGAELLTGGVMVPPPGIGQPGAYIMYSGPEALLDRHRETLRVLGDTT
YVGADVGLSNLYYQAQLYLFWSTLTAYLHSIAMLQSAGVSAEQFRPFATETVTSL
GVDGPMGFLRILAEEADAGHSPGGENSMLMMAVGADHMVEAAEAAGIDTMGP
RALRDLFWRTVNAGHGADGLGSVIEVVRKGA
(IR00020)
SEQ ID 5
MHHHHHHSATTNTTSADGVAGPGGPGGRPPVTVLGLGQMGAAIAGALLAAGHP
VTVWNRTPGKAAPLVEQGAVLAGSVAEAVAASPLVLSVVLDYPALYGILDPEPDA
LKGRALVNLTTGTPEQAGEAAEWAARHGVDYLDGAIMTTPPGVGTREVMFLYS
GDRAVFDAHHAALDVLGEPLHLGTEPGLAALYDVNLLGLMWATMAGWLHGTAV
VGAEGTRAVDFTEVAIRWLGTVNNFIRRYAAQVDEGVYPGDDATVDVQIAVVEH
QLHAAEARGVDNRLPELLKTLMLEANAKGHGQDSFGSVVEVLRKGARR
(IR00032)
SEQ ID 6
MHHHHHHRHLSVIGLGAMGSALATTLLKAGHPVTVWNRSAAKAAPLQALGATLA
PSVGAAIAASDITLVCVDNYAVSQLLLDEASDAVAGKLLVQLSTGSPQGARALES
WSHARGARYLDGAILCFPAQIGTSDASIICSGASAAFSEAEPVLSLLAPTLDHVAE
AVGAAAAQDCAVAAYFAGGLLGALHGALICEAEGLPVAKVCAQFSELSPILGGDV
AHLGKTLASGDFDHPYASLKTWSAAISRLAGHATDAGIDSRFPRFAADLFEEGVA
QGFGQQEVSALIKVLRARNGAAQ
(IR00033)
SEQ ID 7
MHHHHHHRHLSVIGLGAMGSALATTLLKAGHPVTVWNRSAAKAAPLQALGATLA
PSVGAAIAASDINLVCVDNYAVSQQLLDEASDAVAGKLLVQLSTGSPQGARALES
WSHARGARYLDGAILCFPDQIGTSDASIICSGASAAFSDAEPVLRLLAPTLDHVAE
AVGAAAAQDCAVAAYFAGGLLGALHGALICEAEGLPIAKVCAQFSELSPILGGDV
AHLGKTLASGDFDHPYASLKTWSAAISRLAGHATDAGIDSRFPRFAADLFEEGVA
QGLGQQEVSALIKVLRARNGAAL
(IR00041)
SEQ ID 8
MHHHHHHMSSVSIFGLGAMGTALASRFLEEKYKVAVWNRSPEKASSLLGKGATL
SHTAVDGINASDLIIICLLDNAAVEATLAGALDHLHGKTIINLTNGTPDQARKLSDRF
VSHGARYVHGGIMATPSMIGSPYALVLYSGSPDAFKAAEGDLSVLAKCVFLGED
AGTASLHDLALLSGMYGLFSGFLHATALVRSSTPAVKFMDLLVPWLGAMTEYTK
GMAKQIDEGKYTSEGSNLAMQLVGIQNIIDASEAQQVSAEFIRPMKEFMQKAVAA
GHGGDDISSLIDFVKST
(IR00049)
SEQ ID 9
MHHHHHHMSGDNRAPVTVIGLGMMGAALAGAYLKAGHPTTVWNRSAAKAAPL
VEQGARRATDVAEAVAASGIVVVCVFDYTVARSLLAPVRDQLAGKVIVNLTSGLP
DEARETAAWAEDAGARYLDGYVMTVPPAVGLPQTLLFYGGDKEIFDAHEETLKV
LGGNSVHLGTDPGIAALYDLALLGILWSTLTSALHGFALVGSENVPAAALMPFAE
SWITHVVLPTVQGAAQQVDAGHYATDISTVELNAMGLPKMIKASEAQGVRADLM
VPIKDFLEKRVADGHGADALASLIEVIRDGDR
(IR00052)
SEQ ID 10
MHHHHHHMSNTKAAQAPVSVIGLGLMGQALAAAFLKAGHPTTVWNRTAAKADQ
LVGEGAALAGSTADAIAASPLVVVCVTDYTAVRELLDPLAGALKGKVLVNLTTGT
STQARETAEWAADKEITYLDGAIMAIPPDIATDAAVLLYSGPKAAFDEHEATLRAL
GAAGTTYLDTDHGLSALYDMSLLGIMWGILNGFLHGAALLGTAEVKATTFAPLAN
TMINVVTEYVTAYAPQIDEGKYPAGDATMTVHQDALEHLAEESETLGINAEMPRF
FKALVDRSVAAGHAESGYAALIEQFRKPAV
(IR00054)
SEQ ID 11
MHHHHHHMKSNSQNEKNGSETTNAVGNRKSVTVIGLGPMGQAMADVFLEYGY
SVTVWNRTSSKADQLVAKGAIRVSTVNEALAANELVILSLTDYNVMYSILEPVSEN
LFGKVLVNLSSDTPEKARKAAKWLEDRGARHITGGVQVPPSGIGKSESYTYYSG
DRVVFEAHRETLEVLTSSDYRGEDPGLAMLYYQIQMDIFWTAMLSYLHALAIANA
NGITAEQFLPYASAMMSSLPKFVEFYTPRLDEGEHPGDVDRLAMGLASVEHVVH
TTQEAGIDIALPATVLEVFRRGMKTGHASDSFTSLIEIFKNSDIRS
(IR00060)
SEQ ID 12
MHHHHHHMSTVSASESPVTVVGLGLMGHALAAAFVAKGHPTTVWNRSAGKAD
DLVAAGATLADSVQAAVEASPLVVVCVSDYDAVHALLDPVGPALAGRTLVNLTTA
SSSQARDTAEWAAKLDATYLDGAILALPQGIGTDEATLLYAGPKAAFEEHQATLA
VLGEAATVYLDEDHGLSALYDMAVLTIMWGVLNSFLHAAALLGTANVKATTFAG
MAATAINVTADYVAAYAPQIDAGEYPATDATVNVHVGGMQHLLEESKALGVNAE
LPRFFLELAGRAVAGGHAEDSYAALIKQFRAPSA
(IR00063)
SEQ ID 13
MHHHHHHMKPTLTVIGAGRMGSALIKAFLQSGYTTTVWNRTKAKSEPLAKLGAH
LADTVRDAVKRSDIIVVNVLDYDTSDQLLRQDEVTRELRGKLLVQLTSGSPALAR
EQETWARQHGIDYLDGAIMATPDFIGQAECALLYSGSAALFEKHRAVLNVLGGAT
SHVGEDVGHASALDSALLFQMWGTLFGTLQALAISRAEGIPLEKTTAFIKLTEPVT
QGAVADVLTRVQQNRLTADAQTLASLEAHNVAFQHLLALCEERNIHRGVADAMY
SVIREAVKAGHGKDDFAILTRFLK
(IR00064)
SEQ ID 14
MHHHHHHMSTPPHTTAGPAAVTVLGLGRMGSALAAAFLAAGHSTTVWNRTPGK
ADELAARGARRAGSVAEAVAAAPLVVVCVADDEAVHQLLDPLDGALAGRTLVNL
TTGTSAQARANAAWAKERGAAFLDGAIMAVPEDIATGDAVLLYSGPRDAFDAYE
EALRVLAPAGTTHLGGDAGLAALHDLALLGIMWGVLNGFLHGAALLGTAGVRAG
DFAPLAARMTTVVAGYVTAAAPEVDAGSYPAGDATLTVHQEAMRHLAEESEALG
VNAELPRFLQLLAGRAVAEGHAESGYSALVEQFRKA
(IR00065)
SEQ ID 15
MHHHHHHMKPHISILGAGRMGSALVKAFLQNEYTTTVWNRTRARCEPLAAAGA
RIADSVRDAVQTASVVIVNVNDYDTSDALLRQDEVTQELRGKVLVQLTSGSPKLA
REQATWARRHGIDYLDGAIMATPDLIGRPDCTLLYAGPKALYDKHQAVLAALGG
NTQHVSEDEGHASALDSAILFQLWGSLFSGLQAAAICRAEGIALDALGPHLEAVA
AMIQFSMKDLLQRIQKEQYGADPQSPATLDTHNVAFQHLLHLCEERNIHHALPKA
MDALIQTARKAGHGQDDFSVLARFLR
(IR00066)
SEQ ID 16
MHHHHHHMSVSVLGLGPMGQALTRALLNANHRTTVWNRTAAKADDVVARGAV
WADDPASAIAAGDLTLVNVVDQQAVDAVLTAAGDAVAGRVIIGLSAGTPDAARRT
ASFVTGAGGAYLDGAIMTPTDTIGTASASVLFSGPRELFDDHREVLSALGTLTWL
GEDVGRAEAFDVALLDLFWTSVSGFVHALSIAKANGVSAVELLPHANGISEILPAI
FTEIAERVESGRHDDASAPVSSVAASLRNLISAARSAGIDAGALETFRDYVDAAV
AAGHGDAEISRITPLGIG
(IR00073)
SEQ ID 17
MHHHHHHMKNDGVTKGSVALLGLGEMGRVLAERLLDAGYPVTVWNRTPGRDT
ALVERGARRAETVREAVTAATTVVTCLFDHASVRETLEPVGADLAGRTLVDLTTT
TPNEARWLGGWAEERGIEHLDGAIMATPSMIGAPEASLLYSGSAEAFGRHRTLF
EVWGSATYDGADHGAASLFDLALLSGMYTMFTGFAHGAAMVTSAGVTAEEFAH
RSARLLSAMTGVFPMTAKVIDEGDYTGPGQSLEWTATALDTIARASAEQGVSPG
PIEMTRALVLAQIEAGYGNENSDRIYEELRAG
(IR00075)
SEQ ID 18
MHHHHHHMAATPTNPSTDSGKTPVTVLGLGAMGRALAGAFLKAGHPTTVWNRS
EHKADELVARGAVRAGSVAEAVAASPLIVVCVVDYEVSHRILEPVGADLAGRVLV
NLTSDTPVRSRRAAEWAAGHGVEYLDGAIMVPTPVIGTPEATVLYSGSRRAFDT
YEETLKALGGKAPFLGTDHGVAAVYDLAMLSFFYSGMAGLAHAFTLAGEEGVPA
TDLAPFLDVITGIFPPIAKGMADDLVGGRLDGAGEGNIVMEAAGIAHIVEASRDRG
VNTDVLDALKALMDRTIAAGHGESEFVRVTEAMRGAYA
(IR00100)
SEQ ID 19
MGSSHHHHHHSSGLVPRGSHMPDNPSTKGRMMRNQQAEHTPVTVIGLGLMGQ
ALAGAFLGAGHPTTVWNRTAAKAEPLVARGAKSAGSVAEAVAASPLVVVCVSDY
DAVHALLDPLDGTALQGRTLVNLTSGTSAQARERAAWADGRGADYLDGAILAGP
AAIGTADAVVLLSGPRSAFDPHASALGGLGAGTTYLGADHGLASLYDAAGLVMM
WSILNGFLQGAALLGTAGVDATTFAPFITQGIGTVADWLPGYARQIDDGAYPADD
AAIDTHLATMEHLIHESEFLGVNAELPRFIKALADRAVADGHGGSGYPALIEQFRT
HSGK
(IR00202)
SEQ ID 20
MGSSHHHHHHSSGLVPRGSMTDTSAKLTLLGLGAMGSALATAWLAADYDITVW
NRTASRAEPLRTLGAQVADTAADAVAANDLVVACLLDDASVRSTLDGVDLTGKD
LINLTTGTPGSGRELAACATARGARYVDGGIMAVPPMIGVPDSGAYVFYSGSAA
AFEAHRDALAVGAGTKFVGEDPGFAALYDVALLSAMTGMFAGVSHAFALVRKEN
IDPREFAGLVSGWITAMSGYAHGIAEHLASGDYTTGVTSNLAMMVEGNATMLRT
AHEQGVSPELLEPFMRLMRQRVDDGHGDEDTTGVIDLLLSRR
(IR00376)
SEQ ID 21
MRSEPAAVTVLGLGSMGTALAGALLKCGHATTVWNRSPHKTGPLAERGATVAA
TPEEAVAASPLVIACVLDYAALHAVLDPVADSLAGKTLVNLTSGSPEQAAEAAAW
ARSHGAHYLDGAIMTTPPGVGSPEMMFLYSGERTVLDTHRPVLASLGDPLYLGT
DPGLASLYDAALLGLMWATMTGWLHGTALVGAEGTPATAFTPVAIRWLSAVTGF
LTTYAPQVDAGHYPGDDATVDVQIAAIDHLIHAAAARGVDNALPGLLKAAMERTR
AAGHGSSSYASVIEVLRKAADAR
(IR00393)
SEQ ID 22
MGSSHHHHHHSSGLVPRGSMGDNRTPVTVIGLGLMGQALAAAFLEAGHTTTVW
NRSAGKAEQLVSQGAVQAATPADAVAASELVVVCLSTYDNMHDVIGSLGESLRG
KVIVNLTSGSSDQGRETAAWAEKQGVEYLDGAIMITPPGIGTETAVLFYAGTQSV
FEKYEPALKLLGGGTTYLGTDHGMPALYDVSLLGLMWGTLNSFLHGVAVVETAG
VGAQQFLPWAHMWLEAIKMFTADYAAQIDAGDGKFPANDATLETHLAALKHLVH
ESEALGIDAELPKYSEALMERVISQGHAKNSYAAVLKAFRKPSE
(IR00395)
SEQ ID 23
MGSSHHHHHHSSGLVPRGSHMDNETAPVTVIGLGLMGRALAGAFLRAGHPTTV
WNRTASKAEQLVAEGARLAPTVGDALEASSVAIVCLTDYEVVHELLGAGEIKLDG
TLLINLTSGDSTQAREAARWAEQRGARYLDGAIMAVPPAIGTAEAMILLSGPQSD
FESHKAMLGALGGTTYLGADHGLASLYDVAGLAMMWSILNAWLQGSALVGTAD
VDAATFTPFAQQLASVVVEWLPGYAEQVDSGSFPAEVSALETDVRAMTHLIEES
EAVGVNAEMPRLFKAIADRSIVAGHGGEQYPVLIEEFRKPRDT
(IR00464)
SEQ ID 24
MTSEQRASVTVLGTGSMGSALARAFLAAGYRTHVWNRSAARTTELVAAGATAH
RDITDAVEASPVIIACLSTFAATHASLSPSTGILAGRDIITLNSGTPAQAREFAQWV
RCAGARFLGGAIKNVPAAVGDRNTLLYFGGDRDVFDAHTGTLRVLGGELDFLGV
ETDLAALYESAVGATLLPALLGFLEGAAMLASRGLPAHTMVPYSVKWLRMIESLL
PVLAEEIDTGNYTLLGSSVELFHNALADDRQLAAESGVDLSWHAPMHDLLRRAV
AEGRGDQSITALIELLTARP
(IR00653)
SEQ ID 25
MPTGTTVSSAVSSPVTPPLAVTVIGLGAMGSALAGAFLDAGHSVTVWNRTPGKG
DALAARGAVVAATAEEAVTASELIVVCLVDYDASEAILTPLAKALSGRTLVNVTAD
VPDRARTAAAWAAEHGIAYLDGAVMVPTAVVGTPAGLLFYSGDPTTFERYEPVL
RALGGRTVHVGDSPDRAAVFDVALLDLFYGAMGAMIHAFALARAHGVPAAEVVP
YMTSIVDLLPEALEAMAGDIDARSYPALTAGLGTMAASVDHIVHAGRAAGIDSAQ
MEGIQRMTDRARELGHADSGWAANYEALINPRTRDRS
(IR00887)
SEQ ID 26
MEGFPMGTNAHTGTATETAGTLAEQRSVTVIGLGPMGRAMADAYLDRGYEVTV
WNRTASRADDLVARGATLAPDVARALTANDLVILSLTDYEAMYAILEPATSALAG
RVLVNLTSDTPDKARAAARWAADHGAVQLTGGVTVPPSGIGRPESSTFYSGPRE
VFDAHRPVLEVVTGKADYRGEDPGLAALYYQMGMVMFWTSMLGYWQAIALADA
NGLAASDILGHAVDTANSLPGFFTFYAGRIDAGAHQGDVDRLAMGLASVEHVLH
TNADAGVDTALPAAVVEIFRRGIKAGHGADSFSSLLELMKKGGES
(IR01134)
SEQ ID 27
MSNPNAAQAPVTVIGLGLMGQALAAAFIKQGHPTTVWNRTPGKAEQLTADGAIL
AQSAGKAISASPLVIVCVSDYDGVHEILGPLQGALAGKVLVNLSTGTSAQARETA
EWAAQRGARYLDGAIMAIPPVIGTDGAVLLYSGPQDSFEEHAATLRALGTPGTTY
LGGDHGLSALHDMALLGLMWGILNGFLHGAALLGTAGVKATTFAPLANQMIKEV
TEYVTAYAPQVDEGKYPATDATLTVHQDALEHLADESKAVGINSELPRFFKALVD
RAAADGRADESYAALIEVFRKEAAA
(IR01136)
SEQ ID 28
MSTLPASGAPVTVIGLGLMGQALAGAFLAKGHPTTVWNRSAAKADDLVARGAVL
ADSVQSAVEASPLVVVCVSDYDAVHQLLGPVGGSLAGRTLVNLTTASSTQARET
AEWAQKLDAAYLDGAILALPHAIGTEEATLLYAGPQATFEEHRATLEVLGAEATVY
LDEDHGLSALYDLSVLSIMWGVLNSFLHGAALLGTANVKASTFAELAVTAINVTAE
YASAYAQQIDAGEYPATDATVNVHVGGMTHLVEESETLGVNAELPRFFLELAKR
AVAAGDAENSYASLIKQFRKPSA
(IR01139)
SEQ ID 29
MSTQQTTVTVLGLGLMGRALAAAFLRAGHPTTVWNRTANKADQLVSEGARQAP
TLVDALAASPLTIICLTDYQAVRELLDTHDLDLGATTLINLTSGDSAQARTAAAWA
ASRGARYLDGAIMAIPSAIGTAEAVILHSGPHADFDSHRPILDALGTVTYLGADHG
LASLYDVAGLAMMWSVLNAWLHGTAMLRTAGVDAAAYAPFAQQIAAGVASWLP
GYAEQIDRGAFPAQVSALETDVRAMAHLLAESAAVGVNTELPELIKAMADRAIAA
GHGAEQYPVLIEEFAKAASAPPR
(IR01421)
SEQ ID 30
MNTNPKRFSNGNHPYDAQADGSILGPVTVIGLGAMGSKLARTFVNNGYVTTVW
NRTPEKAAELVGLGASGTTDIADAIAASPLLIVCVLDYDAARHILEPMREKLSGKV
VVNLTTGTPEEARTMGTWMRTQGAEYLDGGIMAVPSLIATPHAVILYSGSQTAYE
TYKQVLERLGTSHYLGSDPALAPLNDLALLSGMYGMFGGFIHAVALAGSEGTKA
ADFTTTLLIPFLQAMIFTMPEMANQIDTGDYSAKDASLAMQAAHDTIGEVSRVQG
VSAETFAPIFELMKRRVEEGYGGDDFASVIELIRKRPSS
(IR01598)
SEQ ID 31
MGSSHHHHHHSSGLVPRGSMHERPPLSVTVLGLGTMGTALASALLDTGHAVTV
WNRTASRATPLITRGAARAGDVGEALAASRVIVTCLLDHASVHDVLDDHASALTG
RTLVNVTNTTPEGSAELASWAASHGADFLDGGIMAVPPQIGTPSAFVLYSGSPS
ALETARPALEAFGDVRYLGSDPSLAALQDTALLSGMYGLFGGILHAFALVRTGGV
TARQFAPVLREWLTGMVGWASSAAERIDDGDHARDVASNLAMQATAYEGFLAV
ARDRGMNPLLLDPLGRLLRRRVAEGFGHEDITGVIDYLTASTNTTGASA
(IR01855)
SEQ ID 32
MKPSISVLGTGRMGSALARALLQAGYRTVVWNRTSEKAEPLAALGATVAPTVRQ
AIDASGIVIVNVSDYAATSTLLRASDVTPGLRGKLIVELTSGTPEGARETSQWTAA
HGARYLDGAILATPDFIGTDAGTILLSGALEPFAANEDVFRALGGNVQHIGTEPGL
ANALDSAVLALMWGALFGGLHAIAVCRAEEIDLGELGRQWAATAPVVEGLVADLI
KRTSAGRFVSDAETLSSISPHYGAFQHLKELMEARRIDRTVVDGYDAIFRRAIASG
HLHDDFAALSQFMGKAEQP
(IR02159)
SEQ ID 33
MPNTNGAQAPVTVIGLGLMGRALAAAFLEAGHPTTVWNRTAAKADRLVAEGAV
RAGSVGDAIAASPLVVVCVTDYAAVRELLGPSAGSLGGKVVANLTTGTSAQARE
TAEWAAGLGARYLDGAIMAIPADIATDAAVLLHSGPKEAFEEHEATLRALGAAGT
TYLDTDPGLSALYDMSLLGIMWGVLNGFLQGAALLGTAKVRATAFAPLANTMIKV
VTEYVTAYAPQIDEGVYPADDATVTVHRDALGHLAEESEKLGVNAELPRFFKALT
DRAATDGHADSSYAALIEQFRKPAA

Preferred are the imine reductases, which have a sequence having at least 80%, preferably at least 90%, more preferably at least 95%, even more preferably at least 98% sequence identity to SEQ IDs 01, 02, 03, 04, 06, 10, 12, 14, 15, 17, 18, 20, 21, 23, 27, 28, 31, 32 and 33.

Even more preferred are imine reductases, which have a sequence having at least 80%, preferably at least 90%, more preferably at least 95%, even more preferably at least 98% sequence identity to SEQ IDs 06, 10, 12, 14, 15, 27 and 32.

The imine reductases used in this invention, which have a sequence having at least 80%, preferably at least 90%, more preferably at least 95%, even more preferably at least 98% sequence identity to any one of SEQ ID NOs. 1 to 33 and which are capable of catalyzing the stereoselective reduction of an aromatic imine into a chiral aromatic amine are novel and therefore represent a further embodiment of the present invention.

The preferences as outlined above apply likewise.

The imine reductases used in the present invention can be obtained by cultivation of host cells in a routine procedure known to the person skilled in the art. That is, a nucleic acid encoding an imine reductase of the invention can be introduced into a suitable host cell(s) to produce the respective protein by recombinant means. These host cells can be any kind of suitable cells, preferably bacterial cells such as E. coli, which can be cultivated in culture. In a first step, this approach may include the cloning of the respective gene into a suitable vector, such as a vector according to the second aspect of the present invention. Vectors are widely used for gene cloning, and can be easily introduced, i.e. transfected, into bacterial cells which have been made transiently permeable to DNA. After the protein has been expressed in the respective host cell, the cells can be harvested and serve as the starting material for the preparation of a cell extract containing the protein of interest. A cell extract containing the protein of interest is obtained by lysis of the cells. Methods of preparing a cell extract by means of either chemical or mechanical cell lysis are well known to the person skilled in the art, and include, but are not limited to, e.g. hypotonic salt treatment, homogenization, or ultrasonification.

A “host cell” can be any kind of organism suitable for application in recombinant DNA technology, and includes, but is not limited to, all sorts of bacterial and yeast strain which are suitable for expressing one or more recombinant protein(s). Examples of host cells include, for example, various Bacillus subtilis or E. coli strains. A variety of E. coli bacterial host cells are known to a person skilled in the art and include, but are not limited to, strains such as DH5-alpha, HB101, MV1190, JM109, JM101, or XL-1 blue which can be commercially purchased from diverse suppliers including, e.g., Stratagene (CA, USA), Promega (WI, USA) or Qiagen (Hilden, Germany). A particularly suitable host cell is also described in the Examples, namely E. coli BL21 (DE3) cells. Bacillus subtilis strains which can be used as a host cell include, e.g., 1012 wild type: leuA8 metB5 trpC2 hsdRM1 and 168 Marburg: trpC2 (Trp-), which are, e.g., commercially available from MoBiTec (Germany).

The term “nucleic acid” as used herein generally relates to any nucleotide molecule which encodes the imine reductase of the invention and which may be of variable length. Examples of a nucleic acid of the invention include, but are not limited to, plasmids, vectors, or any kind of DNA and/or RNA fragment(s) which can be isolated by standard molecular biology procedures, including, e.g. ion-exchange chromatography. A nucleic acid of the invention may be used for transfection or transduction of a particular cell or organism.

Nucleic acid molecule may be in the form of RNA, such as mRNA or cRNA, or in the form of DNA, including, for instance, cDNA and genomic DNA e.g. obtained by cloning or produced by chemical synthetic techniques or by a combination thereof. The DNA may be triple-stranded, double-stranded or single-stranded. Single-stranded DNA may be the coding strand, also known as the sense strand, or it may be the non-coding strand, also referred to as the anti-sense strand.

Nucleic acid molecule as used herein also refers to, among other, single- and double-stranded DNA, DNA that is a mixture of single- and double-stranded RNA, and RNA that is a mixture of single- and double-stranded regions, hybrid molecules comprising DNA and RNA that may be single-stranded or, more typically, double-stranded, or triple-stranded, or a mixture of single- and double-stranded regions. In addition, nucleic acid molecule as used herein refers to triple-stranded regions comprising RNA or DNA or both RNA and DNA.

Additionally, the nucleic acid may contain one or more modified bases. Such nucleic acids may also contain modifications e.g. in the ribose-phosphate backbone to increase stability and half-life of such molecules in physiological environments.

Thus, DNAs or RNAs with backbones modified for stability or for other reasons are 33 “nucleic acid molecule” as that feature is intended herein. Moreover, DNAs or RNAs comprising unusual bases, such as inosine, or modified bases, such as tritylated bases, to name just two examples, are nucleic acid molecule within the context of the present invention. It will be appreciated that a great variety of modifications have been made to DNA and RNA that serve many useful purposes known to those of skill in the art. The term nucleic acid molecule as it is employed herein embraces such chemically, enzymatically or metabolically modified forms of nucleic acid molecule, as well as the chemical forms of DNA and RNA characteristic of viruses and cells, including simple and complex cells, inter alia.

Furthermore, the nucleic acid molecule encoding the imine reductase of the invention can be functionally linked, using standard techniques such as standard cloning techniques, to any desired sequence, such as a regulatory sequence, leader sequence, heterologous marker sequence or a heterologous coding sequence to create a fusion protein.

The nucleic acid may be originally formed in vitro or in a cell in culture, in general, by the manipulation of nucleic acids by endonucleases and/or exonucleases and/or polymerases and/or ligases and/or recombinases or other methods known to the skilled practitioner to produce the nucleic acids.

The nucleic acid may be comprised in an expression vector, wherein the nucleic acid is operably linked to a promoter sequence capable of promoting the expression of the nucleic acid in a host cell.

In a preferred embodiment the nucleic acid codes for an imine reductase, wherein the imine reductase consists of or comprises an amino acid sequence that is at least 90%, 95% or 98% identical to the amino acid sequence of any of SEQ ID NO: 1 to 33.

As used herein, the term “vector” generally refers to any kind of nucleic acid molecule that can be used to express a protein of interest in a cell. In particular, the vector can be any plasmid or vector known to the person skilled in the art which is suitable for expressing a protein in a particular host cell including, but not limited to, mammalian cells, bacterial cell, and yeast cells. A vector may also be a nucleic acid which encodes an imine reductase of the invention, and which is used for subsequent cloning into a respective vector to ensure expression. Plasmids and vectors for protein expression are well known in the art, and can be commercially purchased from diverse suppliers including, e.g., Promega 35 (Madison, WI, USA), Qiagen (Hilden, Germany), Invitrogen (Carlsbad, CA, USA), or MoBiTec (Germany). Methods of protein expression are well known to the person skilled in the art and are, e.g., described by Sambrook et al., 2000.

The vector may additionally include nucleic acid sequences that permit it to replicate in the host cell, such as an origin of replication, one or more therapeutic genes and/or selectable marker genes and other genetic elements known in the art such as regulatory elements directing transcription, translation and/or secretion of the encoded protein. The vector may be used to transduce, transform or infect a cell, thereby causing the cell to express nucleic acids and/or proteins other than those native to the cell. The vector optionally includes materials to aid in achieving entry of the nucleic acid into the cell, such as a viral particle, liposome, protein coating or the like. Numerous types of appropriate expression vectors are known in the art for protein expression, by standard molecular biology techniques. Such vectors are selected from among conventional vector types including insects, e.g., baculovirus expression, or yeast, fungal, bacterial or viral expression systems. Other appropriate vectors, of which numerous types are known in the art, can also be used for this purpose. Methods for obtaining such vectors are well-known (Sambrook et al, 2000).

As detailed above, the nucleic acid which encodes an imine reductase of the invention is operably linked to a sequence which is suitable for driving the expression of a protein in a host cell, in order to ensure expression of the protein. However, it is encompassed within the present invention that the claimed vector may represent an intermediate product, which is subsequently cloned into a suitable vector to ensure expression of the protein. The vector of the present invention may further comprise all kind of nucleic acid sequences, including, but not limited to, polyadenylation signals, splice donor and splice acceptor signals, intervening sequences, transcriptional enhancer sequences, translational enhancer sequences, drug resistance gene(s) or alike. Optionally, the drug resistance gene may be operably linked to an internal ribosome entry site (IRES), which might be either cell cycle-specific or cell cycle-independent.

The term “operably linked” as used herein generally means that the gene elements are arranged as such that they function in concert for their intended purposes, e.g. in that transcription is initiated by the promoter and proceeds through the DNA sequence encoding the imine reductase of the present invention. That is, RNA polymerase transcribes the sequence encoding the imine reductase into mRNA, which is then spliced and translated into a protein. The term “promoter sequence” as used in the context of the present invention generally refers to any kind of regulatory DNA sequence operably linked to a downstream coding sequence, wherein said promoter is capable of binding RNA polymerase and initiating transcription of the encoded open reading frame in a cell, thereby driving the expression of said downstream coding sequence. The promoter sequence of the present invention can be any kind of promoter sequence known to the person skilled in the art, including, but not limited to, constitutive promoters, inducible promoters, cell cycle-specific promoters, and cell type-specific promoters. Moreover, the present invention also comprises a host cell comprising the imine reductase of the present invention, the nucleic acid encoding the imine reductase or the vector containing the nucleic acid encoding the imine reductase.

### EXAMPLES

### Methods

### Synthesis of the Pre-Formed Imines:

The imines were synthesized according to one of the two procedures described below.

### Procedure A:

The pre-formed imine substrates were synthesized according to a slightly modified procedure described in Carlson et al. 1992

Reactions were carried out in a 100 mL three-necked round-bottom flask with a magnetic stirring bar, a 50 mL dropping funnel, and an argon inlet. Dry toluene (10 mL) as well as the required amine (9 mmol, 3 equiv.) were added to the flask. TiCl₄ (1 M in methylene chloride/toluene, 3 mmol, 1 equiv.) was added via the dropping funnel over a period of 10 minutes while cooling with an ice bath. The reaction was then stirred for 10 minutes at room temperature before the addition of the required carbonyl (3 mmol, 1 equiv.). The reaction was stirred overnight at room temperature.

For the work up, formed titanium complexes were precipitated by the addition of diethyl ether (20 mL). After 20 minutes of further stirring the reaction was filtered through celite and washed with diethyl ether. The solvent was removed under reduced pressure. Products were purified via flash column chromatography.

### Procedure B:

The pre-formed imine substrates were synthesized according to the procedure described in Yasukawa et al. 2022

Reactions were carried out in a stirring hot plate, placed in a glovebox using a 20 mL glass screw top vial equipped with a magnetic stirring bar and molecular sieves (5 Å, 2 mm diameter, 1 g). Toluene (5 mL), the required carbonyl (3 mmol, 1 equiv.) and the required amine (3-15 mmol, 1-5 equiv.) were added to the vial and the reaction was stirred at 120° C. overnight or at 60° C. for 72 h.

For the work up, the reaction was cooled to room temperature and removed from the glovebox. The mixture was filtered through celite and solvent was removed under reduced pressure.

### Enzyme Preparation:

For imine reductase gene acquisition and construction of expression vectors, imine reductases (IREDs) open reading frames were designed and synthesized for expression in Escherichia coli (E. coli), based on the reported amino acid sequence of the imine reductase and the codon optimization was performed using the algorithm of Twist Bioscience (South San Francisco, U.S.A.). A stop codon in the end was added in all cases. Restriction sites for the subsequent cloning in the vector of interest, pET-21(+), were added in the nucleotide sequence; NdeI restriction was added in the 5′ end, and the XhoI restriction sequence in the 3′ end. The vector contains the coding sequence for ampicillin resistance. According to the cloning strategy, the expression is under the control of a lac promoter. Resulting plasmids were transformed into E. coli BL21(DE3) using standard methods.

Plasmids from Twist Bioscience were resuspended in sterile water. Inoculation in E. coli BL21(DE3) cells was achieved by thermal heating (42° C. for 45 s). A pre-culture was incubated overnight at 37° C., on a Luria Bertani medium agar plate, containing 100 μg/mL ampicillin. A single microbial colony was picked and incubated overnight at 37° C. in liquid LB medium containing 100 μg/mL ampicillin. The liquid LB culture was used to inoculate a Terrific Broth (TB) medium containing 100 μg/mL ampicillin. Following 3.5 hours incubation at 37° C., isopropyl-β-D-thiogalactoside (IPTG) was added in the TB culture at a final concentration of 1 mM to induce the expression of the IREDs. Incubation continued overnight at 20° C. Cells were harvested via centrifugation (4° C., 8000 rpm, 20 min) and the supernatant was discarded. The cells were resuspended in Potassium phosphate buffer (100 mM, pH 7). They were then sonicated (02:30 min, 2 sec ON, 4 sec OFF, amplitude 20%). Sonicated cells were centrifuged (4° C., 17000 rpm, 20 min), the lysate was frozen at −20° C. and subsequently lyophilized. The lyophilized enzyme preparation was stored at −20° C.

### Reaction Setup:

Small scale biotransformations using clarified cell free extract or lyophilized cell free extract of E. coli BL21(DE3) with overexpressed imine reductases (IREDs) as biocatalysts were performed in a volume of 0.5 mL. Reactions were performed at pH 8.0 in bicine buffer, at 100 mM concentration. Pre-formed imines were dissolved in methanol at 200 mM concentration and then dispensed in the reactions, the final concentration of the pre-formed imine was 20 mM. NADP⁺ (1 mM), sodium phosphite (200 mM) and phosphite dehydrogenase (4 g/L), or glucose (200 mM) and glucose dehydrogenase (0.1 g/L), and IRED (4 g/L) were dissolved from stock solutions prepared in water. The reactions were incubated in an Eppendorf thermoshaker at 25° C. and under vigorous stirring for 18 to 24 hours.

To determine the conversion of the reaction, the peaks in the GC chromatograph were compared to the peaks from known standards of the imine, the product amine, the ketone and the amine resulting from the imine hydrolysis.

Preparative scale biotransformations using clarified cell free extract or lyophilized cell free extract with overexpressed IREDs as a biocatalyst were performed in a total volume of 30 mL. Unless stated otherwise, reactions using IREDs were performed at 30° C. in a 100 mL flask with mechanical stirrer, including pH and temperature sensor. Reactions were performed at pH 8.0, which was kept constant using 1 M NaOH solution and a titration system from Metrohm (Titrando 902, Touch control). Concentrations, listed in the following reaction description are related to the starting volume (30 mL).

The preparative reaction was prepared in an aqueous solution containing 100 mM bicine buffer pH 8 to which were added lyophilized IRED (8-4 g/L), NADP⁺ (1 mM), sodium phosphite (200 mM) and phosphite dehydrogenase (8-4 g/L), or glucose dehydrogenase (200 mM) and glucose dehydrogenase (0.1 g/L). Finally, the pre-formed imine (final concentration in the reaction 100 mM-200 mM) was dissolved in methanol (10% v/v) and it was transferred to the reaction.

The pH was kept constant by addition of a 1 M NaOH.

At different reaction times, samples were taken and analyzed by GC to determine the progression of the reaction. Enantiomeric excess was analyzed by GC or HPLC. The GC and HPLC methods used are described below.

At the end of the reaction, the primary or the secondary amine was isolated by increasing the pH of the reaction by addition of Na₂CO₃, followed by liquid-liquid extraction with an organic solvent (i.e. ethyl acetate). Subsequently, the solvent was evaporated under reduced pressure and the product amine was isolated as a solid or an oil.

### GC Method:

A sample of the reaction (500 μL) was basified with Na₂CO₃ 2M (200 μL), then ethyl acetate was used for extraction (500 μL, twice) and the mixture was shaken vigorously. The reaction solution was centrifuged at 14,100×g for 5 min and the organic layer was transferred to a glass vial for analysis.

### Conversion Determination Using GC Analysis:

### Method 1

Column: DB-1701 (30 m×250 μm×0.25 μm)
Inlet temperature: 250° C.
Detector temperature: 300° C.
Injection volume: 1 μL
Split ratio: 90:1
Oven temperature program: 40° C., 2 min; 10° C./min to 300° C.; 300° C., 2 min
Total run time: 30 min

### Method 2

Column: RTX5 amine (30 m×250 μm×0.25 μm)
Inlet temperature: 200° C.
Detector temperature: 321° C.
Injection volume: 1 μL
Split ratio: 20:1
Oven temperature program: 100° C., 0 min; 40° C./min to 320° C.; 320° C., 2 min
Total run time: 6.5 min

### Method 3 (Chiral)

Column: BGB-178 (30 m×250 μm×0.25 μm)
Inlet temperature: 200° C.
Detector temperature: 321° C.
Injection volume: 1 μL
Split ratio: 40:1
Oven temperature program: 85° C., 0 min; 1.3° C./min to 115° C.; 115° C., 0 min
Total run time: 15 min

### HPLC Method:

A sample of the reaction (500 μL) was basified with Na₂CO₃ 2M (200 μL), then ethyl acetate was used for extraction (500 μL, twice) and the mixture was shaken vigorously. The reaction solution was centrifuged at 14,100×g for 5 min. The supernatant was used for enantiomeric excess determination using HPLC.

### Enantiomeric Excess Determination Using HPLC Analysis

### Method 1:

Column: Daicel Chiralcel OD-H (4.6 mm×250 mm×5 μm)
Eluents: 99/1 n-heptane/2-propanol
Column temperature: 20° C.
Additives: 0.1% DEA
Flow rate: 0.5 mL/min
Total run time: 60 min
DAD: 218 nm

### Method 2:

Column: Daicel Chiralpak IC column (4.6 mm×250 mm×5 μm)
Eluents: 90/10 n-heptane/2-propanol
Column temperature: 20° C.
Additives: 0.1% DEA
Flow rate: 0.5 mL/min
Total run time: 60 min
DAD: 218 nm

### Method 3:

Column: Daicel Chiralpak AD-3R column (2.1 mm×250 mm×3 μm)
Eluents: 90/10 methanol/water
Column temperature: 25° C.
Flow rate: 0.7 mL/min
Total run time: 8 min
DAD: 245 nm

### Method 4:

Column: Daicel Chiralpak AD-3R column (2.1 mm×250 mm×3 μm)
Eluents: 65/35 20 mM ammonium formate pH 9.0+5% acetonitrile/20 mM ammonium formate+90% acetonitrile
Column temperature: 40° C.
Flow rate: 1.5 mL/min
Total run time: 15 min
DAD: 218 nm

### Method 5:

Column: Daicel Chiralpak AD-3R column (2.1 mm×250 mm×3 μm)
Eluents: 60/40 20 mM ammonium formate pH 9.0+5% acetonitrile/20 mM ammonium formate+90% acetonitrile
Column temperature: 40° C.
Flow rate: 1.2 mL/min
Total run time: 15 min
DAD: 218 nm

### Method 6:

Column: Daicel Chiralpak IG-3 column (4.6 mm×250 mm×3 μm)
Eluents: 70/30 20 mM ammonium formate/acetonitrile
Column temperature: 35° C.
Flow rate: 1.0 mL/min
Total run time: 12 min
DAD: 210 nm

### Method 7:

Column: Daicel Chiralpak IG-3 column (4.6 mm×250 mm×3 μm)
Eluents: 70/30 20 mM ammonium formate/acetonitrile
Column temperature: 30° C.
Flow rate: 1.3 mL/min
Total run time: 13 min
DAD: 210 nm

### Method 8:

Column: Daicel Chiralpak AD-3R column (2.1 mm×250 mm×3 μm)
Eluents: 50/50 20 mM ammonium formate pH 9.0+5% acetonitrile/20 mM ammonium formate+90% acetonitrile
Column temperature: 40° C.
Flow rate: 1.5 mL/min
Total run time: 25 min
DAD: 210 nm

### Method 9:

Column: Daicel Chiralpak IG-3 column (4.6 mm×250 mm×3 μm)
Eluents: 50/50 20 mM ammonium formate/acetonitrile
Column temperature: 30° C.
Flow rate: 1.3 mL/min
Total run time: 12 min
DAD: 210 nm

### Method 10:

Column: Daicel Chiralpak IG-3 column (4.6 mm×250 mm×3 μm)
Eluents: 55/45 20 mM ammonium formate/acetonitrile
Column temperature: 30° C.
Flow rate: 1.3 mL/min
Total run time: 18 min
DAD: 210 nm

Methods used in Example 1:

| Example number | Conversion method | Enantiomeric excess method |
|---|---|---|
| 1 | GC method 1 | Not determined |
| 2 | GC method 2 | HPLC method 9 |
| 3 | GC method 1 | HPLC method 1 |
| 4 | GC method 1 | HPLC method 10 |
| 5 | GC method 1 | HPLC method 9 |
| 6 | GC method 1 | HPLC method 2 |
| 7 | GC method 2 | GC method 3 |
| 8 | GC method 2 | HPLC method 7 |
| 9 | GC method 2 | HPLC method 3 |
| 10 | GC method 2 | HPLC method 5 |
| 11 | GC method 2 | HPLC method 4 |
| 12 | GC method 2 | Not determined |
| 13 | GC method 2 | HPLC method 8 |
| 14 | GC method 2 | HPLC method 6 |

### Comparison Example

### Reductive Amination by the Selected IREDs

According to the scientific rationale (Sharma et al., 2018), the reductive amination of an aromatic ketone and a primary amine in the presence of an enzyme with imine reductase activity is expected to go via the intermediary aromatic imine which is reduced in the presence of the enzyme and provide the chiral aromatic amine.

This example shows the attempt of the direct reductive amination of the aromatic ketone precursor acetophenone and the primary amine 3-phenylpropylamine and the measurement of the conversion into the desired chiral secondary aromatic amine 3-phenyl-N-(1-phenylethyl)propan-1-amine.

Small scale biotransformations using clarified cell free extract of E. coli BL21(DE3) with overexpressed different imine reductases (IREDs) as biocatalysts were performed in a volume of 0.5 mL. Reactions were performed at pH 8.0 in bicine buffer. NADP⁺ (1 mM), sodium phosphite (200 mM), phosphite dehydrogenase (4 g/L) and IRED (4 g/L) were dissolved from stock solutions prepared in water. Acetophenone and 3-phenylpropylamine were dissolved in methanol at 200 mM concentration and then dispensed in the reactions. The final concentration of the substrate was 20 mM. The reactions were incubated in an Eppendorf thermoshaker at 25° C. and under vigorous stirring for 24 hours.

The reactions were quenched by adding Na₂CO₃ 2 M (200 μL) and then the reactions were extracted with ethyl acetate (500 μL, twice). The organic layer was transferred to a glass vial for analysis.

To determine the conversion in the reaction, the peaks in the GC chromatograph were compared to the peaks from known standards of the imine, the secondary amine and the ketone and the amine obtained from the imine hydrolysis.

TABLE A
Results of the screening of IREDs for the direct reductive amination of
acetophenone with 3-phenylpropylamine to 3-phenyl-N-(1-phenyl-
ethyl)propan-1-amine

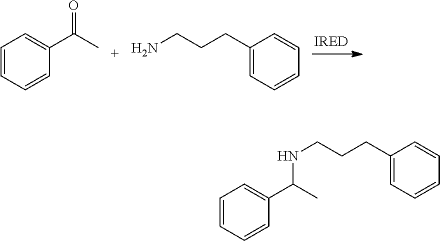

| | Conversion |
|---|---|
| IRED | [Peak Area %] |
| IR00464 | 0 |
| IR00033 | 0 |
| IR00032 | 0 |
| IR00064 | 0 |
| IR01134 | 0 |
| IR00887 | 0 |
| IR00060 | 0 |
| IR00052 | 0 |
| IR01855 | 0 |
| IR00065 | 0 |

Representative reactions from the compounds listed in Example 1 were also tested for the direct reductive amination in parallel to the imine reduction reaction. For the examples reported below, direct reductive amination did not yield the amine product.

### Example 1

### Screening of the Reduction of Different Pre-Formed Imines by IREDs

Small scale biotransformations using clarified cell free extract of E. coli BL21(DE3) with overexpressed different imine reductases (IREDs) as biocatalysts were performed in a volume of 0.5 mL. Reactions were performed at pH 8.0 in bicine buffer, at 100 mM concentration. Pre-formed imines were dissolved in methanol at 200 mM concentration and then dispensed in the reactions, the final concentration of the preformed imine was 20 mM. NADP⁺ (1 mM), sodium phosphite (200 mM), phosphite dehydrogenase (4 g/L), or glucose (200 mM) and GDH (0.1 (g/L), and IRED (4 g/L) were dissolved from stock solutions prepared in water. The reactions were incubated in an Eppendorf thermoshaker at 25° C. and under vigorous stirring for 18 to 24 hours.

In the small scale biotransformations, the reactions were quenched by adding Na₂CO₃ 2M (200 μL) and then the reactions were extracted with ethyl acetate (500 μL, twice). The organic layer was transferred to a glass vial for analysis.

To determine the conversion in the reaction, the peaks in the GC chromatograph were compared to the peaks from known standards of the imine, the secondary amine and the ketone and the amine obtained from the imine hydrolysis.

A selection of the best results of the imine reduction by IREDs is summarized below. For enantioselectivity determination, in absence of a standard with known stereochemical assignment, it was indicated the prevalent enantiomer by the number 1 or 2, which refers to the order of elution of the enantiomers in the chiral method.

TABLE 1
Results of the screening of IREDs for the reduction of 1-phenyl-N-(3-
phenylpropyl)ethan-1-imine to 3-phenyl-N-(1-phenylethyl)propan-
1-amine

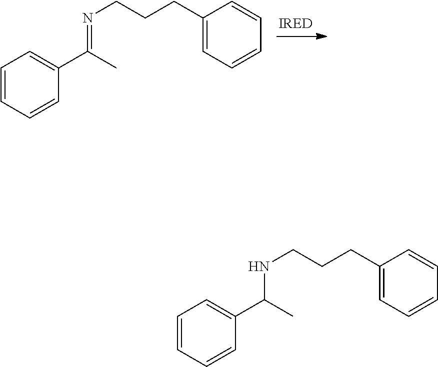 [SMILES: C/C(=N\CCCc1ccccc1)c1ccccc1 ; CC(NCCCc1ccccc1)c1ccccc1]

| | Conversion | |
|---|---|---|
| IRED | [Peak Area %] | ee [%] |
| IR00464 | 74.4 | Not determined |
| IR00033 | 73.0 | Not determined |
| IR00032 | 72.5 | Not determined |
| IR00064 | 67.8 | Not determined |
| IR01134 | 43.5 | Not determined |
| IR00887 | 42.6 | Not determined |
| IR00060 | 42.2 | Not determined |
| IR00052 | 41.9 | Not determined |
| IR01855 | 39.3 | Not determined |
| IR00065 | 22.7 | Not determined |

TABLE 2
Results of the screening of IREDs for the reduction of N-1-diphenyl-
ethanimine to N-(1-phenylethyl)aniline

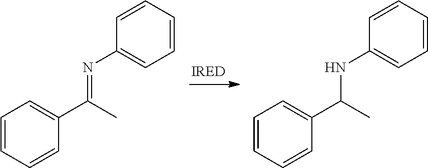

| | Conversion | |
|---|---|---|
| IRED | [Peak Area %] | ee [%] |
| IR00395 | 88.0 | 97 (2) |
| IR00005 | 88.0 | 99 (1) |
| IR01855 | 81.8 | 98 (2) |
| IR00065 | 80.5 | 85 (2) |
| IR00060 | 75.2 | 16 (1) |
| IR00041 | 73.9 | >99 (2) |
| IR00049 | 73.7 | 90 (2) |
| IR00376 | 68.3 | 99 (2) |
| IR01139 | 67.0 | >99 (2) |
| IR00393 | 60.2 | 98 (2) |
| IR01134 | 59.3 | 68 (2) |
| IR00010 | 51.3 | 91 (1) |
| IR00066 | 38.6 | 57 (1) |
| IR00020 | 38.4 | 95 (2) |
| IR00014 | 32.2 | 31 (2) |
| IR00015 | 30.1 | 99 (1) |
| IR00032 | 20.5 | 27 (1) |

TABLE 3
Results of the screening of IREDs for the reduction of N-(4-methoxy-
phenyl)-1-(naphthalen-1-yl)ethan-1-imine to 4-methoxy-N-[1-(1-
naphthyl)ethyl]aniline

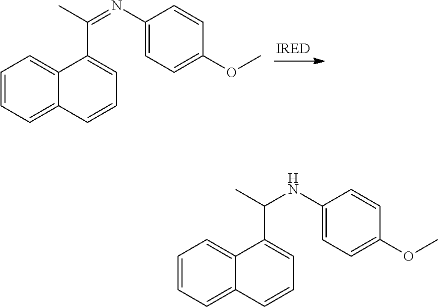 [SMILES: COc1ccc(/N=C(/C)c2cccc3ccccc23)cc1 ; COc1ccc(NC(C)c2cccc3ccccc23)cc1]

| | Conversion | |
|---|---|---|
| IRED | [Peak Area %] | ee [%] |
| IR00060 | 99.6 | 99 (2) |
| IR01855 | 82.2 | 11 (2) |
| IR00052 | 76.3 | 21 (2) |
| IR00014 | 44.5 | 95 (2) |
| IR00033 | 30.8 | 92 (2) |
| IR00065 | 30.0 | 83 (2) |
| IR00064 | 27.0 | 12 (2) |
| IR00032 | 19.8 | 90 (2) |
| IR01134 | 11.2 | 12 (2) |

TABLE 4
Results of the screening of IREDs for the reduction of methyl-2-
phenyl-2-(phenylimino)acetate to methyl 2-anilino-2-phenyl-acetate

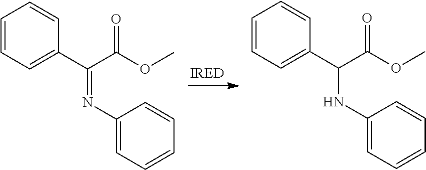 [SMILES: COC(=O)/C(=N\c1ccccc1)c1ccccc1 ; COC(=O)C(Nc1ccccc1)c1ccccc1]

| | Conversion | |
|---|---|---|
| IRED | [Peak Area %] | ee [%] |
| IR00060 | 99.6 | 94 (2) |
| IR00064 | 62.1 | 74 (2) |
| IR00005 | 56.5 | 54 (2) |
| IR00052 | 51.6 | 21 (2) |
| IR00065 | 41.3 | 16 (2) |
| IR01134 | 39.6 | 5 (2) |
| IR00395 | 27.7 | 34 (1) |
| IR00653 | 22.2 | 33 (2) |
| IR00014 | 21.4 | 85 (2) |
| IR00063 | 14.2 | 65 (2) |
| IR00066 | 12.3 | 25 (2) |

TABLE 5
Results of the screening of IREDs for the reduction of N-(4-meth-
oxyphenyl)-2,2-dimethyl-1-phenylpropan-1-imine to N-(2,2-
dimethyl-1-phenylpropyl)-4-methoxy-aniline

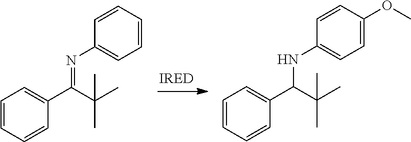 [SMILES: CC(C)(C)/C(=N\c1ccccc1)c1ccccc1 ; COc1ccc(NC(c2ccccc2)C(C)(C)C)cc1]

| | Conversion | |
|---|---|---|
| IRED | [Peak Area %] | ee [%] |
| IR00052 | >99.9 | 99 (2) |
| IR01134 | 99.9 | 94 (2) |
| IR00064 | 97.5 | 97 (2) |
| IR00060 | 97.0 | 99 (2) |
| IR00100 | 88.2 | >99 (2) |
| IR00063 | 71.3 | >99 (2) |
| IR00065 | 57.4 | 98 (2) |
| IR00014 | 38.0 | 99 (2) |
| IR00393 | 24.9 | 21 (2) |
| IR00049 | 24.4 | 36 (2) |
| IR00376 | 23.8 | 98 (1) |

TABLE 6
Results of the screening of IREDs for the reduction of N-(4-methoxy-
phenyl)tetralin-1-imine to N-(4-methoxyphenyl)tetralin-1-amine

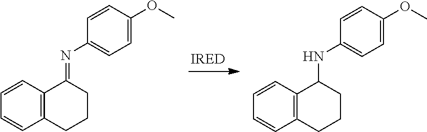 [SMILES: COc1ccc(/N=C2\CCCc3ccccc32)cc1 ; COc1ccc(NC2CCCc3ccccc32)cc1]

| | Conversion | |
|---|---|---|
| IRED | [Peak Area %] | ee [%] |
| IR02239 | 61.4 | >99 (1) |
| IR00395 | 48.1 | >99 (1) |
| IR00060 | 21.2 | 99 (1) |

TABLE 7
Results of the screening of IREDs for the reduction
of N-methyl-1-phenyl-ethanimine to N-methyl-1-
phenyl-ethanamine

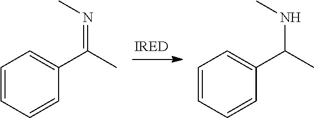

| | Conversion | |
|---|---|---|
| IRED | [Peak Area %] | ee [%] |
| IR00052 | 56.3 | 84 (1) |
| IR00073 | 43.6 | >99 (1) |
| IR00032 | 38.1 | >99 (1) |
| IR00005 | 29.9 | >99 (1) |
| IR00064 | 27.5 | >99 (1) |
| IR01134 | 24.4 | >99 (1) |
| IR00202 | 21.0 | >99 (1) |
| IR01589 | 14.8 | 61 (2) |
| IR02159 | 17.8 | >99 (1) |
| IR01855 | 13.9 | 42 (1) |
| IR01136 | 10.5 | >99 (1) |

TABLE 8
Results of the screening of IREDs for the reduction of N-allyl-1-
phenyl-ethanimine to N-(1-phenylethyl)prop-2-en-1-amine

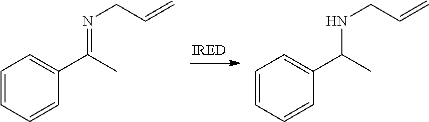

| | Conversion | |
|---|---|---|
| IRED | [Peak Area %] | ee [%] |
| IR00005 | 61.0 | >99 (1) |
| IR00032 | 57.6 | >99 (1) |
| IR01136 | 56.2 | 97 (1) |
| IR00073 | 56.5 | >99 (1) |
| IR00064 | 54.1 | 94 (1) |
| IR00052 | 53.9 | 79 (1) |
| IR00060 | 42.2 | 93 (1) |
| IR01598 | 41.9 | 78 (1) |
| IR01134 | 33.6 | 92 (1) |
| IR00202 | 32.9 | 79 (2) |
| IR02159 | 31.4 | >99 (1) |
| IR00065 | 27.8 | 84 (1) |
| IR01855 | 23.2 | 81 (2) |
| IR01139 | 22.4 | 96 (2) |
| IR00010 | 19.0 | 76 (1) |
| IR00015 | 12.7 | 14 (2) |

TABLE 9
Results of the screening of IREDs for the reduction of N-phenyl-1-
(3-pyridyl)ethanimine to N-[1-(3-pyridyl)ethyl]aniline

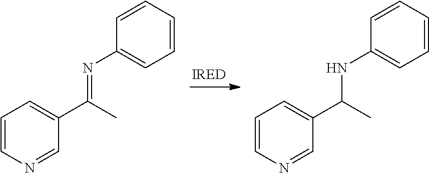

| | Conversion | |
|---|---|---|
| IRED | [Peak Area %] | ee [%] |
| IR00005 | 77.7 | >99 (1) |

TABLE 10
Results of the screening of IREDs for the reduction of 1-phenyl-N-(3-
pyridyl)ethanimine to N-(1-phenylethyl)pyridin-3-amine

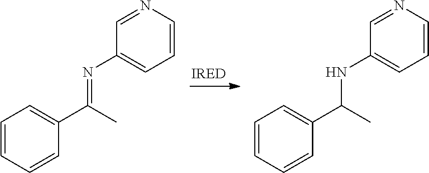

| | Conversion | |
|---|---|---|
| IRED | [Peak Area %] | ee [%] |
| IR01855 | 16.9 | 99 (1) |
| IR00065 | 13.1 | 90 (1) |

TABLE 11

| Results of the screening of IREDs for the reduction of | | |
|---|---|---|
| N-benzyl-1-phenyl-ethanimine to N-benzyl-1- | | |
| phenyl-ethanamine | | |
| 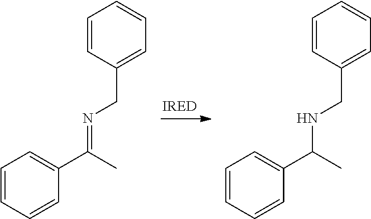 [SMILES: C/C(=N\Cc1ccccc1)c1ccccc1 ; CC(NCc1ccccc1)c1ccccc1] | | |
| | Conversion | |
| IRED | [Peak Area %] | ee [%] |
| IR00052 | 61.6 | 96 (2) |
| IR01855 | 60.6 | >99 (2) |
| IR01139 | 55.9 | >99 (2) |
| IR00064 | 51.8 | 49 (2) |
| IR00395 | 47.5 | >99 (2) |
| IR00065 | 38.3 | >99 (2) |
| IR01134 | 20.7 | 83 (2) |
| IR00202 | 19.5 | 43 (2) |
| IR02159 | 17.5 | 97 (2) |
| IR01136 | 10.4 | >99 (2) |

TABLE 12

| Results of the screening of IREDs for the reduction of diphenyl- | |
|---|---|
| methanimine to diphenylmethanamine | |
| 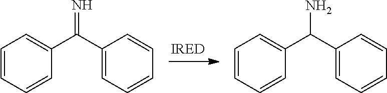 [SMILES: N=C(c1ccccc1)c1ccccc1 ; NC(c1ccccc1)c1ccccc1] | |
| | Conversion |
| IRED | [Peak Area %] |
| IR00005 | 21.3 |

TABLE 13
Results of the screening of IREDs for the reduction of 1-(4-chloro-
phenyl)-N-phenyl-ethanimine to N-[1-(4-chlorophenyl)-
ethyl]aniline

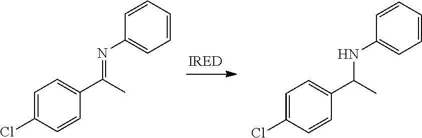 [SMILES: C/C(=N\c1ccccc1)c1ccc(Cl)cc1 ; CC(Nc1ccccc1)c1ccc(Cl)cc1]

| | Conversion | |
|---|---|---|
| IRED | [Peak Area %] | ee [%] |
| IR01136 | 25.8 | 89 (1) |
| IR00005 | 13.5 | >99 (2) |
| IR01136 | 12.8 | 79 (1) |

TABLE 14
Results of the screening of IREDs for the reduction of N-
tert-butyl-1-phenyl-ethanimine to 2-methyl-N-(1-phenyl-
ethyl)propan-2-amine

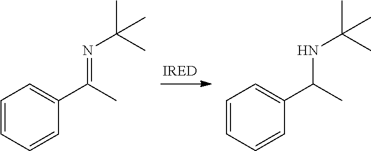

| | Conversion | |
|---|---|---|
| IRED | [Peak Area %] | ee [%] |
| IR00010 | 77.1 | 98 (1) |
| IR00015 | 41.1 | >99 (1) |
| IR00075 | 39.5 | >99 (1) |
| IR00064 | 35.9 | 94 (1) |
| IR00160 | 26.7 | 86 (1) |
| IR00066 | 22.7 | >99 (1) |
| IR00054 | 17.3 | >99 (1) |
| IR00052 | 14.2 | 39 (1) |
| IR00065 | 12.4 | 83 (2) |
| IR00032 | 11.8 | >99 (1) |

### Example 2

### Optimization of Reaction Temperature

For a selection of enzymes (IR00005, IR00395, IR01139), the process parameters were tested in further detail to determine the optimal reaction conditions. In particular, the reaction temperature was tested between 2° and 40° C.

Small scale biotransformations using clarified cell free extract of E. coli BL21(DE3) with overexpressed imine reductases (IREDs) as biocatalysts were performed in a volume of 0.5 mL. Reactions were performed at pH 8.0 in bicine buffer, at 100 mM concentration. N-1-diphenylethanimine was dissolved in methanol at 200 mM concentration and then dispensed in the reactions, the final concentration of the pre-formed imine was 20 mM. NADP⁺ (1 mM), glucose (200 mM) and glucose dehydrogenase (0.1 g/L), and IRED (4 g/L) were dissolved from stock solutions prepared in water. The reactions were incubated in an Eppendorf thermoshaker at 20, 25, 30, 35 and 40° C. and under vigorous stirring for 18 hours.

The reactions were quenched by adding Na₂CO₃ 2 M (200 μL) and then the reactions were extracted with ethyl acetate (500 μL, twice). The organic layer was transferred to a glass vial for analysis.

To determine the conversion in the reaction, the peaks in the GC chromatograph were compared to the peaks from known standards of the imine, the secondary amine and the ketone and the amine obtained from the imine hydrolysis.

The results of the temperature screening were as follows:

TABLE 15
Results of the temperature screening of selected IREDs for the
reduction of N-1-diphenylethanimine to N-(1-phenylethyl)aniline

| | Conversion [Peak Area %] | | |
|---|---|---|---|
| Temperature | IR00005 | IR00395 | IR01139 |
| 20 | 83.1 | 71.3 | 91.7 |
| 25 | 84.6 | 73.8 | 89.5 |
| 30 | 86.8 | 65.0 | 78.5 |
| 35 | 88.4 | 7.0 | 18.4 |
| 40 | 83.9 | 13.3 | 16.0 |

### Example 3

### Optimization of Reaction pH

For a selection of enzymes (IR00005, IR00395, IR01139), the process parameters were tested in further detail to determine the optimal reaction conditions. In particular, the reaction pH was tested between the values of 6.5 and 9.0.

Small scale biotransformations using clarified cell free extract of E. coli BL21(DE3) with overexpressed imine reductases (IREDs) as biocatalysts were performed in a volume of 0.5 mL. Reactions were performed at pH 6.5, 7.0, 7.5, 8.0, 8.5 and 9.0 in bicine buffer, at 100 mM concentration. N-1-diphenylethanimine was dissolved in methanol at 200 mM concentration and then dispensed in the reactions, the final concentration of the pre-formed imine was 20 mM. NADP⁺ (1 mM), glucose (200 mM) and glucose dehydrogenase (0.1 g/L), and IRED (4 g/L) were dissolved from stock solutions prepared in water. The reactions were incubated in an Eppendorf thermoshaker at 30° C. and under vigorous stirring for 18 hours.

The reactions were quenched by adding Na₂CO₃ 2 M (200 μL) and then the reactions were extracted with ethyl acetate (500 μL, twice). The organic layer was transferred to a glass vial for analysis.

To determine the conversion in the reaction, the peaks in the GC chromatograph were compared to the peaks from known standards of the imine, the secondary amine and the ketone and the amine obtained from the imine hydrolysis.

The results of the pH screening were as follows:

TABLE 16
Results of the pH screening of selected IREDs for the reduction
of N-1-diphenylethanimine to N-(1-phenylethyl)aniline

| | Conversion [Peak Area %] | | |
|---|---|---|---|
| pH | IR00005 | IR00395 | IR01139 |
| 6.5 | 79.1 | 71.5 | 85.6 |
| 7.0 | 87.2 | 84.0 | 94.5 |
| 7.5 | 91.5 | 87.0 | 94.4 |
| 8.0 | 86.8 | 65.0 | 78.5 |
| 8.5 | 72.6 | 4.1 | 12.4 |
| 9.0 | 40.2 | 0.6 | 2.5 |

### Example 4

### Optimization of Cosolvent

For a selection of enzymes (IR00005, IR00395, IR01139), the process parameters were tested in further detail to determine the optimal reaction conditions. In particular, the reaction was tested in presence of several cosolvents at 10% (v/v) concentration.

Small scale biotransformations using clarified cell free extract of E. coli BL21(DE3) with overexpressed imine reductases (IREDs) as biocatalysts were performed in a volume of 0.5 mL. Reactions were performed at pH 8.0 in bicine buffer, at 100 mM concentration. N-1-diphenylethanimine was dissolved in one of the following solvents (methanol, DMSO, acetonitrile, glycerol, tert-methyl butyl ether, toluene, cyclopentyl methyl ether, isopropyl acetate, isoamyl acetate, Cyrene™ and cyclohexane) at 200 mM concentration and then dispensed in the reactions, the final concentration of the pre-formed imine was 20 mM. NADP⁺ (1 mM), glucose (200 mM) and glucose dehydrogenase (0.1 g/L), and IRED (4 g/L) were dissolved from stock solutions prepared in water. The reactions were incubated in an Eppendorf thermoshaker at 30° C. and under vigorous stirring for 18 hours.

The reactions were quenched by adding Na₂CO₃ 2 M (200 μL) and then the reactions were extracted with ethyl acetate (500 μL, twice). The organic layer was transferred to a glass vial for analysis.

To determine the conversion in the reaction, the peaks in the GC chromatograph were compared to the peaks from known standards of the imine, the secondary amine and the ketone and the amine obtained from the imine hydrolysis.

The results of the cosolvent screening were as follows:

TABLE 17
Results of the cosolvent screening of selected IREDs for the reduction
of N-1-diphenylethanimine to N-(1-phenylethyl)aniline

Conversion [Peak Area %]

| Cosolvent | IR00005 | IR00395 | IR01139 |
|---|---|---|---|
| Acetonitrile | 34.4 | 0.4 | 1.0 |
| CPME | 12.0 | 1.0 | 0.0 |
| Cyclohexane | 44.8 | 46.4 | 76.5 |
| Cyrene ™ | 81.9 | 56.9 | 74.8 |
| DMSO | 59.5 | 40.2 | 82.3 |
| Isoamyl Acetate | 11.9 | 2.6 | 0.8 |
| Isopropyl Acetate | 0.7 | 1.2 | 1.1 |
| Methanol | 90.1 | 65.8 | 61.0 |
| MTBE | 25.8 | 1.3 | 2.6 |
| Toluene | 0.9 | 1.7 | 10.2 |

For a selection of enzymes (IR00005, IR00395, IR01139), the process parameters were tested in further detail to determine the optimal reaction conditions. In particular, the reaction was tested in presence of a selection of cosolvents at 10, 20 and 30% (v/v) concentration.

Small scale biotransformations using clarified cell free extract of E. coli BL21(DE3) with overexpressed imine reductases (IREDs) as biocatalysts were performed in a volume of 0.5 mL. Reactions were performed at pH 8.0 in bicine buffer, at 100 mM concentration. N-1-diphenylethanimine was dissolved in one of the following solvents (methanol, DMSO, Cyrene™ and cyclohexane) at 200 mM concentration and then dispensed in the reactions, the final concentration of the pre-formed imine was 20 mM. The final concentration of the solvent was adjusted to 10, 20 or 30% (v/v). NADP⁺ (1 mM), glucose (200 mM) and glucose dehydrogenase (0.1 g/L), and IRED (4 g/L) were dissolved from stock solutions prepared in water. The reactions were incubated in an Eppendorf thermoshaker at 30° C. and under vigorous stirring for 18 hours.

The reactions were quenched by adding Na₂CO₃ 2 M (200 μL) and then the reactions were extracted with ethyl acetate (500 μL, twice). The organic layer was transferred to a glass vial for analysis.

To determine the conversion in the reaction, the peaks in the GC chromatograph were compared to the peaks from known standards of the imine, the secondary amine and the ketone and the amine obtained from the imine hydrolysis.

The results of the cosolvent screening were as follows:

TABLE 18
Results of selected cosolvent concentration screening
of selected IREDs for the reduction of N-1-diphenylethanimine
to N-(1-phenylethyl)aniline

Conversion [Peak Area %]

| Cosolvent | IR00005 | IR00395 | IR01139 |
|---|---|---|---|
| Cyclohexane 10% | 44.8 | 46.4 | 76.5 |
| Cyclohexane 20% | 71.9 | 13.6 | 50.4 |
| Cyclohexane 30% | 25.6 | 18.1 | 45.6 |
| Cyrene ™ 10% | 81.9 | 56.9 | 74.8 |
| Cyrene ™ 20% | 69.3 | 0.6 | 0.8 |
| Cyrene ™ 30% | 0.3 | 0.0 | 0.6 |
| DMSO 10% | 59.5 | 40.2 | 82.3 |
| DMSO 20% | 73.1 | 45.5 | 63.4 |
| DMSO 30% | 55.8 | 23.2 | 24.3 |
| Methanol 10% | 90.1 | 65.8 | 61.0 |
| Methanol 20% | 71.3 | 2.3 | 3.6 |
| Methanol 30% | 1.2 | 0.3 | 0.3 |

### Example 5

### Preparative Scale Biotransformation with IRED and methyl2-phenyl-2-(phenylimino)acetate to methyl 2-anilino-2-phenyl-acetate

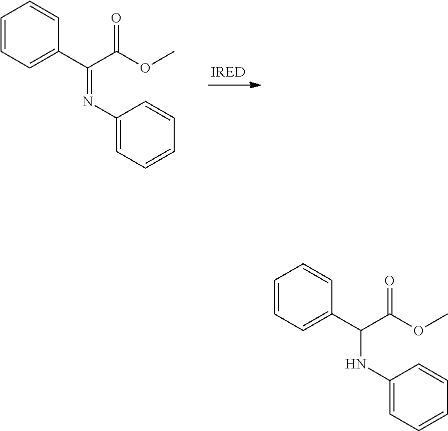 [SMILES: COC(=O)/C(=N\c1ccccc1)c1ccccc1 ; COC(=O)C(Nc1ccccc1)c1ccccc1]

The preparative scale biotransformation of methyl2-phenyl-2-(phenylimino)acetate to methyl 2-anilino-2-phenyl-acetate was performed using lyophilized cell free extract of IR00060 as a biocatalyst. This reaction was performed on 3 mmol scale (781 mg, 23.9 g/L) at 30° C. and 8 g/L IR00060 in 30 mL volume.

Methyl2-phenyl-2-(phenylimino)acetate (781 mg, 3 mmol, 100 mM) was dissolved in 3 mL methanol, which was added to a solution of bicine buffer (100 mM, pH 8.0) containing lyophilized IR00060 (8 g/L), lyophilised phosphite dehydrogenase (4 g/L), NADP⁺ (1 mM), Na₂HPO₃ (100 mM). The reaction was stirred at 30° C. under 120 rpm shaking for 24 hours.

Saturated Na₂CO₃ solution (15 mL) was then added, and the product was extracted with ethyl acetate (2×45 mL). The combined organic phases were dried with Na₂SO₄. The product was purified via flash column chromatography [Biotage® Sfär 25 g; cyclohexane/EtOAc; gradient (EtOAc)=0% (2 CV), 0-20% (20 CV), 20-25% (2 CV)].

The purified amine product was obtained in an isolated yield of 76%, forming preferentially the second eluting enantiomer (94% ee). The optical rotatory power was determined as [α]²⁰_{D}=−98.3° (c=1.3, CHCl₃) which corresponds to the (R)-enantiomer according to literature (Zhang et al., 2014).

Methyl 2-anilino-2-phenyl-acetate: ¹H NMR (300 MHz, CDCl₃) δ ppm 7.55-7.48 (m, 2H), 7.43-7.28 (m, 3H), 7.20-7.07 (m, 2H), 6.78-6.66 (m, 1H), 6.64-6.53 (m, 2H), 5.10 (s, 1H), 3.74 (s, 3H).

### Example 6

### Preparative Scale Biotransformation with IRED and N-1-diphenylethanimine to N-(1-phenylethyl)aniline

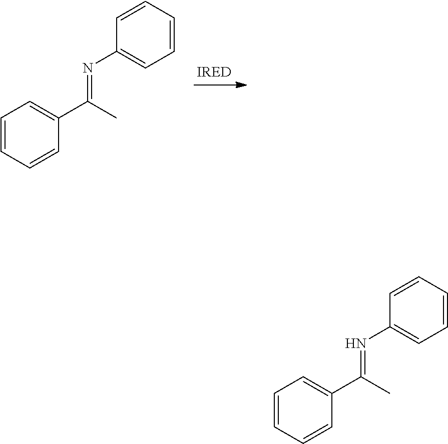

The preparative scale biotransformation of N-1-diphenylethanimine to N-(1-phenylethyl)aniline was performed using lyophilized cell free extract of IR01139 as a biocatalyst. This reaction was performed on 2.5 mmol scale (500 mg, 20 g/L) at 25° C. and 10 g/L IR01139 in 25 mL volume.

N-(1-phenylethyl)aniline (500 mg, 2.5 mmol, 100 mM) was dissolved in 2.5 mL DMSO, which was added to a solution of bicine buffer (100 mM, pH 7.2) containing lyophilized IR01139 (10 g/L), lyophilised glucose dehydrogenase (0.1 g/L), NADP⁺ (1 mM), glucose (200 mM). The reaction was stirred at 25° C. for 18 hours. pH was maintained constant during the course of the reaction using a pH stat.

After 18 hours, the conversion estimated by GC showed that the product N-(1-phenylethyl)aniline accounted for 95% of the total peak areas of the starting materials.

The reaction pH was adjusted to 11 by addition of NaOH 1 M and the product was extracted with ethyl acetate (2×25 mL) and filtered through Dicalite. The combined organic phases were dried with Na₂SO₄ and the solvent was removed under reduced pressure. Isolated yield for the product was 57% (290 mg isolated product, purity 97%, enantiomeric excess not determined).

N-(1-phenylethyl)aniline: ¹H NMR (600 MHz, CDCl₃) δ ppm 7.37-7.41 (m, 2H), 7.32-7.35 (m, 2H), 7.22-7.26 (m, 1H), 7.08-7.13 (m, 2H), 6.66 (tt, J=7.3, 1.1 Hz, 1H), 6.51-6.56 (m, 2H), 4.51 (q, J=6.7 Hz, 1H), 4.04 (br s, 1H), 1.54 (d, J=6.7 Hz, 3H)

### Example 7

### Preparative Scale Biotransformation with IRED and N-phenyl-1-(3-pyridyl)ethanimine to N-[1-(3-pyridyl)ethyl]aniline

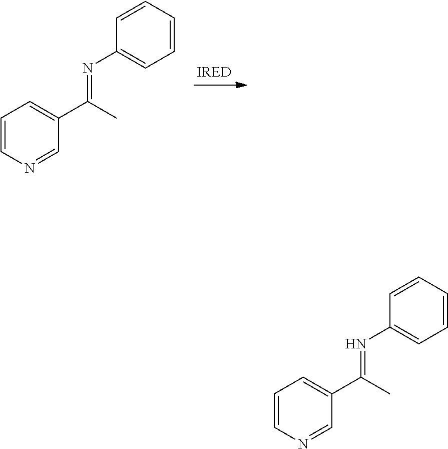

The preparative scale biotransformation of N-phenyl-1-(3-pyridyl)ethanimine to N-[1-(3-pyridyl)ethyl]aniline was performed using lyophilized cell free extract of IR00005 as a biocatalyst. This reaction was performed on 2.5 mmol scale (392.5 mg, 15.7 g/L) at 25° C. and 8 g/L IR00005 in 25 mL volume.

N-phenyl-1-(3-pyridyl)ethanimine (392.5 mg, 2.0 mmol, 80 mM) was dissolved in 2.5 mL methanol, which was added to a solution of bicine buffer (100 mM, pH 8.0) containing lyophilized IR00005 (8 g/L), lyophilised glucose dehydrogenase (0.1 g/L), NADP⁺ (1 mM), glucose (200 mM). The reaction was stirred at 25° C. for 24 hours. pH was maintained constant during the course of the reaction using a pH stat.

Already after 6 hours, the conversion estimated by GC showed that the product N-[1-(3-pyridyl)ethyl]aniline accounted for 91% of the total peak areas of the starting materials. The conversion remained unchanged until the reaction was stopped after 24 hours.

The reaction pH was adjusted to 11 by addition of NaOH 1 M and the product was extracted with ethyl acetate (3×30 mL) and filtered through Dicalite. The combined organic phases were dried with Na₂SO₄ and the solvent was removed under reduced pressure. Isolated yield for the product was 85% (356 mg isolated product, purity 95%, enantiomeric excess 98%).

N-[1-(3-pyridyl)ethyl]aniline: ¹H NMR (600 MHz, CDCl₃) δ ppm 8.67 (d, J=2.2 Hz, 1H), 8.51 (dd, J=4.8, 1.7 Hz, 1H), 7.69-7.72 (m, 1H), 7.23-7.27 (m, 1H), 7.12 (dd, J=8.6, 7.3 Hz, 2H), 6.68-6.72 (m, 1H), 6.50-6.54 (m, 2H), 4.52-4.61 (m, 1H), 3.96-4.15 (m, 1H), 1.57 (d, J=6.7 Hz, 3H)

### Example 8

### Preparative Scale Biotransformation with IRED and N-(4-methoxyphenyl)-2,2-dimethyl-1-phenylpropan-1-imine to N-(2,2-dimethyl-1-phenyl-propyl)-4-methoxy-aniline

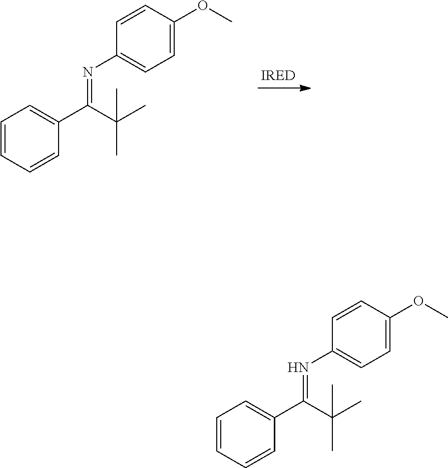 [SMILES: COc1ccc(/N=C(/c2ccccc2)C(C)(C)C)cc1 ; COc1ccc(/N=C(/c2ccccc2)C(C)(C)C)cc1]

The preparative scale biotransformation of N-(4-methoxyphenyl)-2,2-dimethyl-1-phenylpropan-1-imine was performed using lyophilized cell free extract of IR00005 as a biocatalyst. This reaction was performed on 2.0 mmol scale (534.7 mg, 21.4 g/L) at 25° C. and 8 g/L IR00060 in 25 mL volume.

N-(2,2-dimethyl-1-phenyl-propyl)-4-methoxy-aniline (392.5 mg, 2.0 mmol, 80 mM) was dissolved in 2.5 mL methanol, which was added to a solution of bicine buffer (100 mM, pH 8.0) containing lyophilized IR00060 (8 g/L), lyophilised glucose dehydrogenase (0.1 g/L), NADP⁺ (1 mM), glucose (200 mM). The reaction was stirred at 25° C. for 24 hours.

After 24 hours, the conversion estimated by GC showed that the product N-(2,2-dimethyl-1-phenyl-propyl)-4-methoxy-aniline accounted for 92% of the total peak areas of the starting materials.

The reaction pH was adjusted to 11 by addition of NaOH 1 M and the product was extracted with ethyl acetate (3×50 mL) and filtered through Dicalite. The combined organic phases were dried with Na₂SO₄ and the solvent was removed under reduced pressure. Isolated yield for the product was 82% (460 mg isolated product, purity 96%, enantiomeric excess not determined).

N-(2,2-dimethyl-1-phenyl-propyl)-4-methoxy-aniline: ¹H NMR (600 MHz, CDCl₃) δ ppm 7.27-7.35 (m, 3H), 7.18-7.24 (m, 1H), 6.61-6.69 (m, 2H), 6.40-6.48 (m, 2H), 4.01 (br s, 1H), 3.94-3.98 (m, 1H), 3.64-3.70 (m, 3H), 0.96-1.04 (m, 9H)

### REFERENCES

Afanasyev, O. I., Kuchuk, E., Usanov, D. L. and Chusov, D., 2019. Reductive amination in the synthesis of pharmaceuticals. Chemical reviews, 119(23), pp. 11857-11911.
Aleku, G. A., France, S. P., Man, H., Mangas-Sanchez, J., Montgomery, S. L., Sharma, M., Leipold, F., Hussain, S., Grogan, G. and Turner, N.J., 2017. A reductive aminase from Aspergillus oryzae. Nature Chemistry, 9(10), pp. 961-969.
Carlson, R., Larsson, U. and Hansson, L., 1992. Efficient synthesis of imines by a modified titanium tetrachloride procedure. Acta Chemica Scandinavica, 46, pp. 1211-1211.
France, S. P., Howard, R. M., Steflik, J., Weise, N. J., Mangas-Sanchez, J., Montgomery, S. L., Crook, R., Kumar, R. and Turner, N.J., 2018. Identification of novel bacterial members of the imine reductase enzyme family that perform reductive amination. Chem Cat Chem, 10(3), pp. 510-514.
Roiban, G. D., Kern, M., Liu, Z., Hyslop, J., Tey, P. L., Levine, M. S., Jordan, L. S., Brown, K. K., Hadi, T., Ihnken, L. A. F. and Brown, M. J., 2017. Efficient biocatalytic reductive aminations by extending the imine reductase toolbox. Chem Cat Chem, 9(24), pp. 4475-4479.
Sambrook, J. F. and Russell, D. W., 2000. Molecular cloning: A laboratory manual, third 39 edition. Cold Spring Harbor Laboratory Press.
Sharma, M., Mangas-Sanchez, J., France, S. P., Aleku, G. A., Montgomery, S. L., Ramsden, J. I., Turner, N. J. and Grogan, G., 2018. A mechanism for reductive amination catalyzed by fungal reductive aminases. ACS Catalysis, 8(12), pp. 11534-11541.
Scheller, P. N., Lenz, M., Hammer, S. C., Hauer, B. and Nestl, B. M., 2015. Imine reductase-catalyzed intermolecular reductive amination of aldehydes and ketones. Chem Cat Chem, 7(20), pp. 3239-3242.
Yasukawa, T., Nakajima, H., Masuda, R., Yamashita, Y. and Kobayashi, S., 2022. Effect of Activation Methods of Molecular Sieves on Ketimine Synthesis. The Journal of Organic Chemistry, 87(21), pp. 13750-13756.
Zhang, X., Staples, R. J., Rheingold, A. L. and Wulff, W. D., 2014. Catalytic asymmetric α-iminol rearrangement: new chiral platforms. Journal of the American Chemical Society, 136(40), pp. 13971-13974.

1. A process for producing a chiral aromatic amine comprising the steps of
a) providing an aromatic imine, and
b) contacting the aromatic imine from step a) with an imine reductase,
thereby stereoselectively reducing the aromatic imine with the imine reductase to a chiral aromatic amine.

2. The process according to claim 1, wherein the chiral aromatic amine is a chiral primary or secondary aromatic amine.

3. The process according to claim 1, wherein the aromatic imine has the formula I 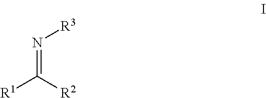
and the chiral aromatic amine has the formula II 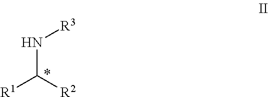
wherein
R¹ is substituted or unsubstituted aryl or heteroaryl,
R² is hydrogen, substituted or unsubstituted C₁₋₆-alkyl or substituted or unsubstituted C₁₋₆-alkoxy carbonyl, or
R¹ and R² together form a carbocycle or heterocycle and
R³ is hydrogen or substituted or unsubstituted aryl or heteroaryl, substituted or
unsubstituted C₁₋₆-alkyl or substituted or unsubstituted C₂₋₆-alkenyl.
and wherein
the substituents are selected from halogen, halogen C₁₋₆-alkyl, C₁₋₆-alkyl, hydroxy, aryl, C₁₋₆-alkyloxy, aryloxy or C₁₋₆-alkoxy carbonyl or aryloxy carbonyl, cyano or nitro.

4. The process according to claim 3,
wherein
R¹ is substituted or unsubstituted aryl or heteroaryl,
R² is hydrogen, C₁₋₆-alkyl or C₁₋₆-alkoxy carbonyl or
R¹ and R² together form a carbocycle or heterocycle and
R³ is hydrogen or substituted or unsubstituted aryl or heteroaryl, substituted or unsubstituted C₁₋₆-alkyl or C₂₋₆-alkenyl,
and wherein
the substituents are selected from C₁₋₆-alkyl, C₁₋₆-alkoxy or aryl.

5. The process according to claim 1, wherein the concentration of the aromatic imine is from 2 mM to 1000 mM.

6. The process according to claim 1, wherein the stereoselective reduction in step b) takes place in the presence of a cofactor regeneration system comprising a cofactor and an enzyme capable of reducing such cofactor.

7. The process according to claim 6, wherein the cofactor is NADP⁺ or NAD⁺ and the enzyme capable of reducing the cofactor is phosphite dehydrogenase or glucose dehydrogenase.

8. The process according to claim 1, wherein the stereoselective reduction in step b) takes place in an aqueous environment having a pH of 5.0 to 10.0 at a temperature of 5° C. to 50° C.

9. The process according to claim 1, wherein the stereoselective reduction in step b) takes place at a sodium phosphite or glucose concentration of 100 mM to 1 M and/or at a phosphite dehydrogenase or glucose dehydrogenase concentration of 0.1 g/L to 10 g/L.

10. The process according to claim 1, wherein the aromatic imine and the imine reductase brought in contact in step b) are incubated for stereoselective reduction for 1 h to 100 h.

11. The process according to claim 1, wherein the imine reductase has a sequence having at least 80% sequence identity to any one of SEQ ID NOs. 1 to 33.

12. An imine reductase having a sequence that has at least 90% sequence identity to any one of SEQ ID NOs. 1 to 33.

13. The process according to claim 1, wherein the chiral aromatic amine has the formula II [chemical structure]


SEQUENCE LISTING (33 sequences)

SEQ ID NO: 1 - protein, 302 aa, synthetic construct
MHHHHHHKTVAVIGLGQMGTTLARLFIEAGMQVRVWNRTRSKAEPLASRGAIVAATAAAAMADAEAVVICVHDYRATHDILSDVAVKSALKGKLLLQLTTGSPQDARDMAELAARIGAGYLDGALQVAPEQMGQPDTTVLVSGSGEDHALARELLAVLGGNVVYLGEDVAAAATMDLATLSYVYGASMGFFQGAALAQAEGLDVGVYGGIVEAMSPSFGAFLRHEGNVIDNGDYAVSQSPLSISIDATGRIEQAMRQKGLRSELPSLIARLLRDAEEAGYGNEEFAAVAKILRGAAEPAPVR

SEQ ID NO: 2 - protein, 298 aa, synthetic construct
MHHHHHHTTTPTVTVLGLGPMGQALSRALLDAGHTVTVWNRTESKAQALRDRGALSAPTPAAAIAASDLALVNVVDHDAVDAILTAAGDAPAGRTVIGLSSDTPDRARRTAKLVGNVGGRYLDGAIMTPIDTIGTRGASILFAGPQALFDEHRGVLDTLGQLTWVGEDHGRAAAFDMALLDLFWTSVGGFGHALMVARANGIEPSELMPHAHGIVGILSPIFTEVAQRVEDDRHSDASASVSSVASSVRHLIAASREAGVDAGLLEAFRGYVDATVAAGHGDDEISRIASEMTTLTRG

SEQ ID NO: 3 - protein, 295 aa, synthetic construct
MHHHHHHTNNATPVSILGLGLMGQALARAFLKAGHPTTVWNRTPGKADQLMAEGAQVAPTAAEAIDASSLTVICVSDYPAMYELLDASDLAGTTLLNLTSGDSAQARQAARWAEQRGAHYLDGAIMAIPQAIGTDDAVILISGAQADADAHRPTLEALGTLTYLGADHGLASLYDVAGLAMMWSVLNAWLQGTALLRTAGVDAATFAPFAQQMAAGVAGWLPGHAQEIDAGSFATEVASLDTHVRTMDHLIEECEAAGINAELPRLIKSMADRSLAAGHGAASYSVLIEEFAKPA

SEQ ID NO: 4 - protein, 298 aa, synthetic construct
MHHHHHHMKAPVTVVGLGPMGKAMAETFLKNGHPTTVWNRTASKAAPLVEQGATLAATPDDALAASGLVVISQTDYKAMYDSLDGAEMKGRVLVNLSSGSPDELRRAAEWAAGKGAELLTGGVMVPPPGIGQPGAYIMYSGPEALLDRHRETLRVLGDTTYVGADVGLSNLYYQAQLYLFWSTLTAYLHSIAMLQSAGVSAEQFRPFATETVTSLGVDGPMGFLRILAEEADAGHSPGGENSMLMMAVGADHMVEAAEAAGIDTMGPRALRDLFWRTVNAGHGADGLGSVIEVVRKGA

SEQ ID NO: 5 - protein, 316 aa, synthetic construct
MHHHHHHSATTNTTSADGVAGPGGPGGRPPVTVLGLGQMGAAIAGALLAAGHPVTVWNRTPGKAAPLVEQGAVLAGSVAEAVAASPLVLSVVLDYPALYGILDPEPDALKGRALVNLTTGTPEQAGEAAEWAARHGVDYLDGAIMTTPPGVGTREVMFLYSGDRAVFDAHHAALDVLGEPLHLGTEPGLAALYDVNLLGLMWATMAGWLHGTAVVGAEGTRAVDFTEVAIRWLGTVNNFIRRYAAQVDEGVYPGDDATVDVQIAVVEHQLHAAEARGVDNRLPELLKTLMLEANAKGHGQDSFGSVVEVLRKGARR

SEQ ID NO: 6 - protein, 298 aa, synthetic construct
MHHHHHHRHLSVIGLGAMGSALATTLLKAGHPVTVWNRSAAKAAPLQALGATLAPSVGAAIAASDITLVCVDNYAVSQLLLDEASDAVAGKLLVQLSTGSPQGARALESWSHARGARYLDGAILCFPAQIGTSDASIICSGASAAFSEAEPVLSLLAPTLDHVAEAVGAAAAQDCAVAAYFAGGLLGALHGALICEAEGLPVAKVCAQFSELSPILGGDVAHLGKTLASGDFDHPYASLKTWSAAISRLAGHATDAGIDSRFPRFAADLFEEGVAQGFGQQEVSALIKVLRARNGAAQ

SEQ ID NO: 7 - protein, 298 aa, synthetic construct
MHHHHHHRHLSVIGLGAMGSALATTLLKAGHPVTVWNRSAAKAAPLQALGATLAPSVGAAIAASDINLVCVDNYAVSQQLLDEASDAVAGKLLVQLSTGSPQGARALESWSHARGARYLDGAILCFPDQIGTSDASIICSGASAAFSDAEPVLRLLAPTLDHVAEAVGAAAAQDCAVAAYFAGGLLGALHGALICEAEGLPIAKVCAQFSELSPILGGDVAHLGKTLASGDFDHPYASLKTWSAAISRLAGHATDAGIDSRFPRFAADLFEEGVAQGLGQQEVSALIKVLRARNGAAL

SEQ ID NO: 8 - protein, 292 aa, synthetic construct
MHHHHHHMSSVSIFGLGAMGTALASRFLEEKYKVAVWNRSPEKASSLLGKGATLSHTAVDGINASDLIIICLLDNAAVEATLAGALDHLHGKTIINLTNGTPDQARKLSDRFVSHGARYVHGGIMATPSMIGSPYALVLYSGSPDAFKAAEGDLSVLAKCVFLGEDAGTASLHDLALLSGMYGLFSGFLHATALVRSSTPAVKFMDLLVPWLGAMTEYTKGMAKQIDEGKYTSEGSNLAMQLVGIQNIIDASEAQQVSAEFIRPMKEFMQKAVAAGHGGDDISSLIDFVKST

SEQ ID NO: 9 - protein, 302 aa, synthetic construct
MHHHHHHMSGDNRAPVTVIGLGMMGAALAGAYLKAGHPTTVWNRSAAKAAPLVEQGARRATDVAEAVAASGIVVVCVFDYTVARSLLAPVRDQLAGKVIVNLTSGLPDEARETAAWAEDAGARYLDGYVMTVPPAVGLPQTLLFYGGDKEIFDAHEETLKVLGGNSVHLGTDPGIAALYDLALLGILWSTLTSALHGFALVGSENVPAAALMPFAESWITHVVLPTVQGAAQQVDAGHYATDISTVELNAMGLPKMIKASEAQGVRADLMVPIKDFLEKRVADGHGADALASLIEVIRDGDR

SEQ ID NO: 10 - protein, 304 aa, synthetic construct
MHHHHHHMSNTKAAQAPVSVIGLGLMGQALAAAFLKAGHPTTVWNRTAAKADQLVGEGAALAGSTADAIAASPLVVVCVTDYTAVRELLDPLAGALKGKVLVNLTTGTSTQARETAEWAADKEITYLDGAIMAIPPDIATDAAVLLYSGPKAAFDEHEATLRALGAAGTTYLDTDHGLSALYDMSLLGIMWGILNGFLHGAALLGTAEVKATTFAPLANTMINVVTEYVTAYAPQIDEGKYPAGDATMTVHQDALEHLAEESETLGINAEMPRFFKALVDRSVAAGHAESGYAALIEQFRKPAV

SEQ ID NO: 11 - protein, 317 aa, synthetic construct
MHHHHHHMKSNSQNEKNGSETTNAVGNRKSVTVIGLGPMGQAMADVFLEYGYSVTVWNRTSSKADQLVAKGAIRVSTVNEALAANELVILSLTDYNVMYSILEPVSENLFGKVLVNLSSDTPEKARKAAKWLEDRGARHITGGVQVPPSGIGKSESYTYYSGDRVVFEAHRETLEVLTSSDYRGEDPGLAMLYYQIQMDIFWTAMLSYLHALAIANANGITAEQFLPYASAMMSSLPKFVEFYTPRLDEGEHPGDVDRLAMGLASVEHVVHTTQEAGIDIALPATVLEVFRRGMKTGHASDSFTSLIEIFKNSDIRS

SEQ ID NO: 12 - protein, 304 aa, synthetic construct
MHHHHHHMSTVSASESPVTVVGLGLMGHALAAAFVAKGHPTTVWNRSAGKADDLVAAGATLADSVQAAVEASPLVVVCVSDYDAVHALLDPVGPALAGRTLVNLTTASSSQARDTAEWAAKLDATYLDGAILALPQGIGTDEATLLYAGPKAAFEEHQATLAVLGEAATVYLDEDHGLSALYDMAVLTIMWGVLNSFLHAAALLGTANVKATTFAGMAATAINVTADYVAAYAPQIDAGEYPATDATVNVHVGGMQHLLEESKALGVNAELPRFFLELAGRAVAGGHAEDSYAALIKQFRAPSA

SEQ ID NO: 13 - protein, 298 aa, synthetic construct
MHHHHHHMKPTLTVIGAGRMGSALIKAFLQSGYTTTVWNRTKAKSEPLAKLGAHLADTVRDAVKRSDIIVVNVLDYDTSDQLLRQDEVTRELRGKLLVQLTSGSPALAREQETWARQHGIDYLDGAIMATPDFIGQAECALLYSGSAALFEKHRAVLNVLGGATSHVGEDVGHASALDSALLFQMWGTLFGTLQALAISRAEGIPLEKTTAFIKLTEPVTQGAVADVLTRVQQNRLTADAQTLASLEAHNVAFQHLLALCEERNIHRGVADAMYSVIREAVKAGHGKDDFAILTRFLK

SEQ ID NO: 14 - protein, 305 aa, synthetic construct
MHHHHHHMSTPPHTTAGPAAVTVLGLGRMGSALAAAFLAAGHSTTVWNRTPGKADELAARGARRAGSVAEAVAAAPLVVVCVADDEAVHQLLDPLDGALAGRTLVNLTTGTSAQARANAAWAKERGAAFLDGAIMAVPEDIATGDAVLLYSGPRDAFDAYEEALRVLAPAGTTHLGGDAGLAALHDLALLGIMWGVLNGFLHGAALLGTAGVRAGDFAPLAARMTTVVAGYVTAAAPEVDAGSYPAGDATLTVHQEAMRHLAEESEALGVNAELPRFLQLLAGRAVAEGHAESGYSALVEQFRKA

SEQ ID NO: 15 - protein, 298 aa, synthetic construct
MHHHHHHMKPHISILGAGRMGSALVKAFLQNEYTTTVWNRTRARCEPLAAAGARIADSVRDAVQTASVVIVNVNDYDTSDALLRQDEVTQELRGKVLVQLTSGSPKLAREQATWARRHGIDYLDGAIMATPDLIGRPDCTLLYAGPKALYDKHQAVLAALGGNTQHVSEDEGHASALDSAILFQLWGSLFSGLQAAAICRAEGIALDALGPHLEAVAAMIQFSMKDLLQRIQKEQYGADPQSPATLDTHNVAFQHLLHLCEERNIHHALPKAMDALIQTARKAGHGQDDFSVLARFLR

SEQ ID NO: 16 - protein, 291 aa, synthetic construct
MHHHHHHMSVSVLGLGPMGQALTRALLNANHRTTVWNRTAAKADDVVARGAVWADDPASAIAAGDLTLVNVVDQQAVDAVLTAAGDAVAGRVIIGLSAGTPDAARRTASFVTGAGGAYLDGAIMTPTDTIGTASASVLFSGPRELFDDHREVLSALGTLTWLGEDVGRAEAFDVALLDLFWTSVSGFVHALSIAKANGVSAVELLPHANGISEILPAIFTEIAERVESGRHDDASAPVSSVAASLRNLISAARSAGIDAGALETFRDYVDAAVAAGHGDAEISRITPLGIG

SEQ ID NO: 17 - protein, 300 aa, synthetic construct
MHHHHHHMKNDGVTKGSVALLGLGEMGRVLAERLLDAGYPVTVWNRTPGRDTALVERGARRAETVREAVTAATTVVTCLFDHASVRETLEPVGADLAGRTLVDLTTTTPNEARWLGGWAEERGIEHLDGAIMATPSMIGAPEASLLYSGSAEAFGRHRTLFEVWGSATYDGADHGAASLFDLALLSGMYTMFTGFAHGAAMVTSAGVTAEEFAHRSARLLSAMTGVFPMTAKVIDEGDYTGPGQSLEWTATALDTIARASAEQGVSPGPIEMTRALVLAQIEAGYGNENSDRIYEELRAG

SEQ ID NO: 18 - protein, 310 aa, synthetic construct
MHHHHHHMAATPTNPSTDSGKTPVTVLGLGAMGRALAGAFLKAGHPTTVWNRSEHKADELVARGAVRAGSVAEAVAASPLIVVCVVDYEVSHRILEPVGADLAGRVLVNLTSDTPVRSRRAAEWAAGHGVEYLDGAIMVPTPVIGTPEATVLYSGSRRAFDTYEETLKALGGKAPFLGTDHGVAAVYDLAMLSFFYSGMAGLAHAFTLAGEEGVPATDLAPFLDVITGIFPPIAKGMADDLVGGRLDGAGEGNIVMEAAGIAHIVEASRDRGVNTDVLDALKALMDRTIAAGHGESEFVRVTEAMRGAYA

SEQ ID NO: 19 - protein, 329 aa, synthetic construct
MGSSHHHHHHSSGLVPRGSHMPDNPSTKGRMMRNQQAEHTPVTVIGLGLMGQALAGAFLGAGHPTTVWNRTAAKAEPLVARGAKSAGSVAEAVAASPLVVVCVSDYDAVHALLDPLDGTALQGRTLVNLTSGTSAQARERAAWADGRGADYLDGAILAGPAAIGTADAVVLLSGPRSAFDPHASALGGLGAGTTYLGADHGLASLYDAAGLVMMWSILNGFLQGAALLGTAGVDATTFAPFITQGIGTVADWLPGYARQIDDGAYPADDAAIDTHLATMEHLIHESEFLGVNAELPRFIKALADRAVADGHGGSGYPALIEQFRTHSGK

SEQ ID NO: 20 - protein, 311 aa, synthetic construct
MGSSHHHHHHSSGLVPRGSMTDTSAKLTLLGLGAMGSALATAWLAADYDITVWNRTASRAEPLRTLGAQVADTAADAVAANDLVVACLLDDASVRSTLDGVDLTGKDLINLTTGTPGSGRELAACATARGARYVDGGIMAVPPMIGVPDSGAYVFYSGSAAAFEAHRDALAVGAGTKFVGEDPGFAALYDVALLSAMTGMFAGVSHAFALVRKENIDPREFAGLVSGWITAMSGYAHGIAEHLASGDYTTGVTSNLAMMVEGNATMLRTAHEQGVSPELLEPFMRLMRQRVDDGHGDEDTTGVIDLLLSRR

SEQ ID NO: 21 - protein, 294 aa, synthetic construct
MRSEPAAVTVLGLGSMGTALAGALLKCGHATTVWNRSPHKTGPLAERGATVAATPEEAVAASPLVIACVLDYAALHAVLDPVADSLAGKTLVNLTSGSPEQAAEAAAWARSHGAHYLDGAIMTTPPGVGSPEMMFLYSGERTVLDTHRPVLASLGDPLYLGTDPGLASLYDAALLGLMWATMTGWLHGTALVGAEGTPATAFTPVAIRWLSAVTGFLTTYAPQVDAGHYPGDDATVDVQIAAIDHLIHAAAARGVDNALPGLLKAAMERTRAAGHGSSSYASVIEVLRKAADAR

SEQ ID NO: 22 - protein, 314 aa, synthetic construct
MGSSHHHHHHSSGLVPRGSMGDNRTPVTVIGLGLMGQALAAAFLEAGHTTTVWNRSAGKAEQLVSQGAVQAATPADAVAASELVVVCLSTYDNMHDVIGSLGESLRGKVIVNLTSGSSDQGRETAAWAEKQGVEYLDGAIMITPPGIGTETAVLFYAGTQSVFEKYEPALKLLGGGTTYLGTDHGMPALYDVSLLGLMWGTLNSFLHGVAVVETAGVGAQQFLPWAHMWLEAIKMFTADYAAQIDAGDGKFPANDATLETHLAALKHLVHESEALGIDAELPKYSEALMERVISQGHAKNSYAAVLKAFRKPSE

SEQ ID NO: 23 - protein, 313 aa, synthetic construct
MGSSHHHHHHSSGLVPRGSHMDNETAPVTVIGLGLMGRALAGAFLRAGHPTTVWNRTASKAEQLVAEGARLAPTVGDALEASSVAIVCLTDYEVVHELLGAGEIKLDGTLLINLTSGDSTQAREAARWAEQRGARYLDGAIMAVPPAIGTAEAMILLSGPQSDFESHKAMLGALGGTTYLGADHGLASLYDVAGLAMMWSILNAWLQGSALVGTADVDAATFTPFAQQLASVVVEWLPGYAEQVDSGSFPAEVSALETDVRAMTHLIEESEAVGVNAEMPRLFKAIADRSIVAGHGGEQYPVLIEEFRKPRDT

SEQ ID NO: 24 - protein, 293 aa, synthetic construct
MTSEQRASVTVLGTGSMGSALARAFLAAGYRTHVWNRSAARTTELVAAGATAHRDITDAVEASPVIIACLSTFAATHASLSPSTGILAGRDIITLNSGTPAQAREFAQWVRCAGARFLGGAIKNVPAAVGDRNTLLYFGGDRDVFDAHTGTLRVLGGELDFLGVETDLAALYESAVGATLLPALLGFLEGAAMLASRGLPAHTMVPYSVKWLRMIESLLPVLAEEIDTGNYTLLGSSVELFHNALADDRQLAAESGVDLSWHAPMHDLLRRAVAEGRGDQSITALIELLTARP

SEQ ID NO: 25 - protein, 310 aa, synthetic construct
MPTGTTVSSAVSSPVTPPLAVTVIGLGAMGSALAGAFLDAGHSVTVWNRTPGKGDALAARGAVVAATAEEAVTASELIVVCLVDYDASEAILTPLAKALSGRTLVNVTADVPDRARTAAAWAAEHGIAYLDGAVMVPTAVVGTPAGLLFYSGDPTTFERYEPVLRALGGRTVHVGDSPDRAAVFDVALLDLFYGAMGAMIHAFALARAHGVPAAEVVPYMTSIVDLLPEALEAMAGDIDARSYPALTAGLGTMAASVDHIVHAGRAAGIDSAQMEGIQRMTDRARELGHADSGWAANYEALINPRTRDRS

SEQ ID NO: 26 - protein, 314 aa, synthetic construct
MEGFPMGTNAHTGTATETAGTLAEQRSVTVIGLGPMGRAMADAYLDRGYEVTVWNRTASRADDLVARGATLAPDVARALTANDLVILSLTDYEAMYAILEPATSALAGRVLVNLTSDTPDKARAAARWAADHGAVQLTGGVTVPPSGIGRPESSTFYSGPREVFDAHRPVLEVVTGKADYRGEDPGLAALYYQMGMVMFWTSMLGYWQAIALADANGLAASDILGHAVDTANSLPGFFTFYAGRIDAGAHQGDVDRLAMGLASVEHVLHTNADAGVDTALPAAVVEIFRRGIKAGHGADSFSSLLELMKKGGES

SEQ ID NO: 27 - protein, 298 aa, synthetic construct
MSNPNAAQAPVTVIGLGLMGQALAAAFIKQGHPTTVWNRTPGKAEQLTADGAILAQSAGKAISASPLVIVCVSDYDGVHEILGPLQGALAGKVLVNLSTGTSAQARETAEWAAQRGARYLDGAIMAIPPVIGTDGAVLLYSGPQDSFEEHAATLRALGTPGTTYLGGDHGLSALHDMALLGLMWGILNGFLHGAALLGTAGVKATTFAPLANQMIKEVTEYVTAYAPQVDEGKYPATDATLTVHQDALEHLADESKAVGINSELPRFFKALVDRAAADGRADESYAALIEVFRKEAAA

SEQ ID NO: 28 - protein, 297 aa, synthetic construct
MSTLPASGAPVTVIGLGLMGQALAGAFLAKGHPTTVWNRSAAKADDLVARGAVLADSVQSAVEASPLVVVCVSDYDAVHQLLGPVGGSLAGRTLVNLTTASSTQARETAEWAQKLDAAYLDGAILALPHAIGTEEATLLYAGPQATFEEHRATLEVLGAEATVYLDEDHGLSALYDLSVLSIMWGVLNSFLHGAALLGTANVKASTFAELAVTAINVTAEYASAYAQQIDAGEYPATDATVNVHVGGMTHLVEESETLGVNAELPRFFLELAKRAVAAGDAENSYASLIKQFRKPSA

SEQ ID NO: 29 - protein, 296 aa, synthetic construct
MSTQQTTVTVLGLGLMGRALAAAFLRAGHPTTVWNRTANKADQLVSEGARQAPTLVDALAASPLTIICLTDYQAVRELLDTHDLDLGATTLINLTSGDSAQARTAAAWAASRGARYLDGAIMAIPSAIGTAEAVILHSGPHADFDSHRPILDALGTVTYLGADHGLASLYDVAGLAMMWSVLNAWLHGTAMLRTAGVDAAAYAPFAQQIAAGVASWLPGYAEQIDRGAFPAQVSALETDVRAMAHLLAESAAVGVNTELPELIKAMADRAIAAGHGAEQYPVLIEEFAKAASAPPR

SEQ ID NO: 30 - protein, 312 aa, synthetic construct
MNTNPKRFSNGNHPYDAQADGSILGPVTVIGLGAMGSKLARTFVNNGYVTTVWNRTPEKAAELVGLGASGTTDIADAIAASPLLIVCVLDYDAARHILEPMREKLSGKVVVNLTTGTPEEARTMGTWMRTQGAEYLDGGIMAVPSLIATPHAVILYSGSQTAYETYKQVLERLGTSHYLGSDPALAPLNDLALLSGMYGMFGGFIHAVALAGSEGTKAADFTTTLLIPFLQAMIFTMPEMANQIDTGDYSAKDASLAMQAAHDTIGEVSRVQGVSAETFAPIFELMKRRVEEGYGGDDFASVIELIRKRPSS

SEQ ID NO: 31 - protein, 319 aa, synthetic construct
MGSSHHHHHHSSGLVPRGSMHERPPLSVTVLGLGTMGTALASALLDTGHAVTVWNRTASRATPLITRGAARAGDVGEALAASRVIVTCLLDHASVHDVLDDHASALTGRTLVNVTNTTPEGSAELASWAASHGADFLDGGIMAVPPQIGTPSAFVLYSGSPSALETARPALEAFGDVRYLGSDPSLAALQDTALLSGMYGLFGGILHAFALVRTGGVTARQFAPVLREWLTGMVGWASSAAERIDDGDHARDVASNLAMQATAYEGFLAVARDRGMNPLLLDPLGRLLRRRVAEGFGHEDITGVIDYLTASTNTTGASA

SEQ ID NO: 32 - protein, 296 aa, synthetic construct
MKPSISVLGTGRMGSALARALLQAGYRTVVWNRTSEKAEPLAALGATVAPTVRQAIDASGIVIVNVSDYAATSTLLRASDVTPGLRGKLIVELTSGTPEGARETSQWTAAHGARYLDGAILATPDFIGTDAGTILLSGALEPFAANEDVFRALGGNVQHIGTEPGLANALDSAVLALMWGALFGGLHAIAVCRAEEIDLGELGRQWAATAPVVEGLVADLIKRTSAGRFVSDAETLSSISPHYGAFQHLKELMEARRIDRTVVDGYDAIFRRAIASGHLHDDFAALSQFMGKAEQP

SEQ ID NO: 33 - protein, 297 aa, synthetic construct
MPNTNGAQAPVTVIGLGLMGRALAAAFLEAGHPTTVWNRTAAKADRLVAEGAVRAGSVGDAIAASPLVVVCVTDYAAVRELLGPSAGSLGGKVVANLTTGTSAQARETAEWAAGLGARYLDGAIMAIPADIATDAAVLLHSGPKEAFEEHEATLRALGAAGTTYLDTDPGLSALYDMSLLGIMWGVLNGFLQGAALLGTAKVRATAFAPLANTMIKVVTEYVTAYAPQIDEGVYPADDATVTVHRDALGHLAEESEKLGVNAELPRFFKALTDRAATDGHADSSYAALIEQFRKPAA